(19) United States
(12) Patent Application Publication
(10) Pub. No.: US 2017/0072042 A1
(43) Pub. Date: Mar. 16, 2017

(54) SALMONELLA CHOLERAESUIS-SALMONELLA TYPHIMURIUM VACCINES

(71) Applicant: Boehringer Ingelheim Vetmedica, Inc., St. Joseph, MO (US)

(72) Inventors: Brian James FERGEN, Ames, IA (US); Dianna M. Murphy JORDAN, Ames, IA (US); Troy James KAISER, St. Joseph, MO (US); Rex Alan SMILEY, St. Joseph, MO (US)

(21) Appl. No.: 15/263,611

(22) Filed: Sep. 13, 2016

Related U.S. Application Data
(60) Provisional application No. 62/219,271, filed on Sep. 16, 2015.

(51) Int. Cl. A61K 39/112

(52) U.S. Cl. CPC A61K 39/0275; A61K 2039/552

(57) ABSTRACT

The present invention relates methods of reducing fecal shedding of animals infected with Salmonella by use of a vaccine or immunogenic composition of Salmonella Choleraesuis-Typhimurium.

### BACKGROUND OF THE INVENTION

A. Field of the Invention

The present invention relates to a Salmonella Choleraesuis (SC)-Salmonella enterica ser Typhimurium (ST) vaccine (SC-ST), which is capable of reducing clinical signs of salmonella infection, including but not limited to shedding.

Salmonella Choleraesuis (SC) and Salmonella enterica ser Typhimurium (ST) have been identified as primary pathogens in swine. ST is a primary cause of enteritis and subclinical production losses in growing or finishing pigs and contributes to environmental and carcass contamination. Due to the zoonotic potential, interventional programs for ST have been established across the world attempting to reduce carcass contamination with the ultimate goal of reducing human salmonellosis cases.

B. Description of the Related Art

Salmonella infections have traditionally been treated using SC vaccines, for example, ENTERISOL SC-54® Salmonella Choleraesuis Vaccine Avirulent Live Culture (Boehringer Ingelheim Vetmedica, Inc.). This product is described in U.S. Pat. No. 5,436,001 and U.S. Pat. No. 5,580,557, both hereby incorporated by reference.

Salmonella Typhimurium isolates include a Salmonella Typhimurium 421/125 of Impfstoffwerk Dessau-Tornau (IDT), Germany. This isolate is used as an active ingredient of SALMOPORC, a live Salmonella vaccine, marketed by IDT Biologika GmbH in Europe. The preferred ST of the invention is described by DE2843295 and its equivalent U.S. Pat. No. 3,856,935, both incorporated by reference.

However, there has not been an effective treatment for Salmonella Typhimurium infections in pigs. The associated shedding of ST causes significant potential for cross-contamination of herds as well as increases the costs of managing disposal of carcasses contaminated with ST.

### SUMMARY OF THE INVENTION

The solution to the above technical problem is achieved by the description and the embodiments characterized in the claims.

Thus, the invention in its different aspects is implemented according to the claims.

The present invention provides immunogenic compositions, vaccines, and related methods that overcome deficiencies in the art. The compositions and methods provide treatment for SC and ST infections in pigs.

The present invention is related to inactivated or avirulent live Salmonella vaccines

The Salmonellae of the present invention can be used for the manufacture of such vaccines. In particular, the invention provides improved Salmonella isolates that have been identified below, or any descendant or progeny of one of the aforementioned isolates.

Immunogenic compositions and vaccines of the invention comprise inactivated or avirulent live Salmonellas and may also include an adjuvant. The vaccine may also include other components, such as preservative(s), stabilizer(s) and antigens against other swine pathogens.

Methods of the invention may also comprise admixing a composition of the invention with a veterinarily acceptable carrier, adjuvant, or combination thereof. Those of skill in the art will recognize that the choice of carrier, adjuvant, or combination will be determined by the delivery route, personal preference, and animal species among others.

Another aspect of the invention contemplates a vaccine for the protection of swine against Salmonella infection, comprising inactivated or live avirulent Salmonella of the present invention and a pharmaceutically acceptable carrier.

Such a vaccine may advantageously further comprise one or more non-Salmonella or Salmonellae that differ from the Salmonella of the present invention, avirulent or inactivated pathogens or antigenic material thereof. For example, the non-Salmonella pathogens may be selected from Pseudorabies virus, Porcine influenza virus, Porcine parvovirus, Transmissible gastroenteritis virus, Escherichia coli, Erysipelothrix rhusiopathiae, Bordetella bronchiseptica, Haemophilus parasuis, Pasteurella multocida, Streptococcus suis, Mycoplasma hyopneumoniae, Porcine Circovirus, including but not limited to Porcine Circovirus Type 2 (PCV2), Porcine Reproductive and Respiratory Syndrome (PRRS) virus and Actinobacillus pleuropneumoniae.

Methods for the treatment or prophylaxis of infections caused by the Salmonella are also disclosed. The method comprises administering an effective amount of the immunogenic composition of the present invention to an animal, specifically a pig or sow (including gilts). The treatment or prophylaxis is selected from the group consisting of reducing signs of diarrhea (shedding), reducing the severity of or incidence of clinical signs of Salmonella infection, reducing the mortality of animals from Salmonella infection, and combinations thereof.

Herein, suitable subjects and subjects in need to which compositions of the invention may be administered include animals in need of either prophylactic or treatment for an infection, disease, or condition. Animals in which the immune response is stimulated by use of compositions or methods of the invention include livestock, such as swine, bovines, goats, and sheep. Preferred animals include porcines (swine), murids, equids, lagomorphs, and bovids. Most preferably, an immune response is stimulated in swine.

The invention provides a method of reducing the incidence of or severity of one or more clinical signs associated with or caused by a Salmonella infection, comprising the step of administering an immunogenic composition of the invention as provided herewith, such that the incidence of or the severity of a clinical sign of the Salmonella infection is reduced by at least 10%, preferably at least 20%, even more preferred at least 30%, even more preferred at least 50%, even more preferred at least 70%, most preferred at least 100% relative to a subject that has not received the immunogenic composition as provided herewith. Such clinical signs include diarrhea shedding and reduction in average daily weight gain.

Preferred routes of administration include intranasal, oral, intradermal, and intramuscular. Administration orally, most preferably in a single dose, is preferred. Oral methods include, but are not limited to direct oral administration or oral gavage, and via drinking water, preferably through use of an automatic whole-barn dosing device. The most preferred oral dosing method is via drinking water via and an automatic whole-barn dosing device. The skilled artisan will recognize that compositions of the invention may also be administered in two or more doses, as well as, by other routes of administration. For example, such other routes include subcutaneously, intracutaneously, intravenously, intravascularly, intraarterially, intraperitnoeally, intrathecally, intratracheally, intracutaneously, intracardially, intralobally, intramedullarly, or intrapulmonarily. Depending on the desired duration and effectiveness of the treatment, the compositions according to the invention may be administered once or several times, also intermittently, for instance on a daily basis for several days, weeks or months and in different dosages.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 illustrates the results of the study of Example 1 showing vaccinated and unvaccinated pigs (placebo) challenged with Salmonella Typhimurium on day 28 of the study. The upper solid line represents the percent of the unvaccinated pigs from which Salmonella Typhimurium was isolated from their feces on the days of the study; the lower dashed line represents the percentage of vaccinated pigs from which ST was isolated.

FIG. 2 illustrates the results of the study of Example 2 and shows the percent positive by Group over Day Post-Challenge.

FIG. 3 shows individual pig data for control pigs.

FIG. 4 shows individual pig data for vaccinated pigs.

FIG. 5 illustrates the fecal shedding Preventive Fractions (PF) by day post-challenge with penalized B-Spline. Note that the PF=0.00.

FIG. 6 illustrates the Salmonella culture results over days post-challenge with logistical modeling by treatment.

### DETAILED DESCRIPTION

The invention provides an inactivated or avirulent live Salmonella vaccine or immunogenic composition that is administered to pigs, to reduce fecal shedding. In addition, there are methods of administration, methods of making the vaccine, assays, and other aspects of this invention described.

### DEFINITIONS

Unless defined otherwise, all technical and scientific terms used herein have the same meaning as is commonly understood by one of skill in the art to which this invention belongs at the time of filing. The meaning and scope of terms should be clear; however, in the event of any latent ambiguity, definitions provided herein take precedent over any dictionary or extrinsic definition. Further, unless otherwise required by context, singular terms shall include pluralities and plural terms shall include the singular. Herein, the use of “or” means “and/or” unless stated otherwise. Furthermore, the use of the term “including”, as well as other forms such as “includes” and “included” is not limiting. All patents and publications referred to herein are incorporated by reference herein.

The practice of the present invention will employ, unless otherwise indicated, conventional techniques of molecular biology, microbiology, recombinant DNA technology, protein chemistry and immunology, which are within the skill of the art. Such techniques are explained fully in the literature. See, e.g., Sambrook, Fritsch & Maniatis, Molecular Cloning: A Laboratory Manual, Vols. I, II and III, Second Edition (1989); DNA Cloning, Vols. I and II (D. N. Glover ed. 1985); Oligonucleotide Synthesis (M. J. Gait ed. 1984); Nucleic Acid Hybridization (B. D. Hames & S. J. Higgins eds. 1984); Animal Cell Culture (R. K. Freshney ed. 1986); Immobilized Cells and Enzymes (IRL press, 1986); Perbal, B., A Practical Guide to Molecular Cloning (1984); the series, Methods In Enzymology (S. Colowick and N. Kaplan eds., Academic Press, Inc.); Protein purification methods—a practical approach (E. L. V. Harris and S. Angal, eds., IRL Press at Oxford University Press); and Handbook of Experimental Immunology, Vols. I-IV (D. M. Weir and C. C. Blackwell eds., 1986, Blackwell Scientific Publications).

It is to be understood that this invention is not limited to particular DNA, polypeptide sequences or process parameters as such may, of course, vary. It is also to be understood that the terminology used herein is for the purpose of describing particular embodiments of the invention only, and is not intended to be limiting. It must be noted that, as used in this specification and the appended claims, the singular forms “a”, “an” and “the” include plural referents unless the content clearly dictates otherwise. Thus, for example, reference to “an antigen” includes a mixture of two or more antigens, reference to “an excipient” includes mixtures of two or more excipients, and the like.

“Salmonella choleraesuis” or “SC”, as used herein refers to an isolate of Salmonella choleraesuis. More preferably, the SC of the present invention comprises Salmonella choleraesuis var. Kuzendor strain 38 PMNa, having an American Type Culture Collection (ATCC) Accession No. 55105. This isolate was deposited under the Budapest Treaty on Oct. 29, 1990 with the ATCC, located at 10801 University Boulevard, Manassas, Va. 20110, United States and assigned accession number 55105. This isolate is distinguished from its wild type parent by the absence of the virulence plasmid exhibited by the parent and by being able to grown on a medium containing d-xylose, which it metabolizes. It also exhibits an overall increased resistance to PMNL killing and to killing by hydrogen peroxide, and it is non-invasive in a Vero cell assay. SC is further described in U.S. Pat. No. 5,436,001 and U.S. Pat. No. 5,580,557, both incorporated by reference.

“Salmonella Typhimurium” or “ST”, as used herein refers to an isolate of Salmonella Typhimurium. More preferably it refers to a Salmonella Typhimurium 421/125 of Impfstoffwerk Dessau-Tornau (IDT), Germany. This isolate is used as an active ingredient of Salmoporc, a live Salmonella vaccine, marketed by Boehringer Ingelheim Vetmedica GmbH. This isolate originates from the wild type Salmonella Typhimurium isolate 415 of the Metschnikov Institute, Moscow. The preferred Salmonella Typhimurium 421/125 isolate was derived from conventional attenuation chemical mutagenesis (using methylnitroguanidine) and is a modification of the isolate 415 having a deletion within the epimerase gene and is described by DE2843295 and its equivalent U.S. Pat. No. 3,856,935, both incorporated by reference.

An “immunogenic or immunological composition or vaccine”, all used interchangeably in this application, refers to a composition of matter that comprises at least one Salmonella of the present invention, or immunogenic portion thereof, that elicits an immunological response in the host of a cellular or antibody-mediated immune response to the composition. In a preferred embodiment of the present invention, an immunogenic composition induces an immune response and, more preferably, confers protective immunity against one or more of the clinical signs of a Salmonella infection, including fecal shedding.

An “immunogenic” or “antigen” as used herein refer to a polypeptide or protein that elicits an immunological response as described herein. This includes cellular and/or humoral immune responses. Depending on the intended function of the composition, one or more antigens may be included be included. An “immunogenic” Salmonella protein or polypeptide includes the full-length sequence of any of the Salmonellae identified herein or analogs or immunogenic fragments thereof. The term “immunogenic fragment” or “immunogenic portion”, used interchangeably in the application, refers to a fragment or truncated and/or substituted form of Salmonella that includes one or more epitopes and thus elicits the immunological response described herein. In general, such truncated and/or substituted forms, or fragments will comprise at least six contiguous amino acids from the full-length Salmonella. More preferably, the truncated or substituted forms, or fragments will have at least 10, more preferably at least 15, and still more preferably at least 19 contiguous amino acids from the full-length Salmonella. Such fragments can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by concurrently synthesizing large numbers of peptides on solid supports, the peptides corresponding to portions of the protein molecule, and reacting the peptides with antibodies while the peptides are still attached to the supports. Such techniques are known and described in the art, see e.g., U.S. Pat. No. 4,708,871; Geysen et al. (1984) Proc. Natl. Acad. Sci. USA 81:3998-4002; and Geysen et al. (1986) Molec. Immunol. 23:709-715. Similarly, conformational epitopes are readily identified by determining spatial conformation of amino acids such as by, e.g., x-ray crystallography and two-dimensional nuclear magnetic resonance. See Epitope Mapping Protocols, supra. Synthetic antigens are also included within the definition, for example, polyepitopes, flanking epitopes, and other recombinant or synthetically derived antigens. See, e.g., Bergmann et al. (1993) Eur. J. Immunol. 23:2777-2781; Bergmann et al. (1996), J. Immunol. 157:3242-3249; Suhrbier, A. (1997), Immunol. and Cell Biol. 75:402-408; and Gardner et al., (1998) 12th World AIDS Conference, Geneva, Switzerland, Jun. 28-Jul. 3, 1998. (The teachings and content of which are all incorporated by reference herein.)

The term “vaccine” as used herein refers to a pharmaceutical composition comprising at least one immunologically active component that induces an immunological response in an animal and possibly but not necessarily one or more additional components that enhance the immunological activity of the active component. A vaccine may additionally comprise further components typical to pharmaceutical compositions. By way of distinction the immunologically active component of a vaccine may comprise complete bacteria particles in either their original form or as avirulent particles in a so called avirulent live vaccine (ALV) or particles inactivated by appropriate methods in a so called killed vaccine (KV). In another form the immunologically active component of a vaccine may comprise appropriate elements of the organisms (subunit vaccines) whereby these elements are generated either by destroying the whole particle or the growth cultures containing such particles and optionally subsequent purification steps yielding the desired structure(s), or by synthetic processes including an appropriate manipulation by use of a suitable system based on, for example, bacteria, insects, mammalian, or other species plus optionally subsequent isolation and purification procedures, or by induction of the synthetic processes in the animal needing a vaccine by direct incorporation of genetic material using suitable pharmaceutical compositions (polynucleotide vaccination). A vaccine may comprise one or simultaneously more than one of the elements described above. The term “vaccine” as understood herein is either a killed or a live, avirulent vaccine for veterinary use comprising antigenic substances and is administered for the purpose of inducing a specific and active immunity against a disease provoked by a salmonella infection, The inactivated or avirulent live Salmonella, confer active immunity that may be transferred passively via maternal antibodies against the immunogens it contains and sometimes also against antigenically related organisms.

As used herein the terms “inactivated” or “killed” are used synonymously. Various physical and chemical methods of inactivation are known in the art. The term “inactivated” refers to a previously virulent or non-virulent bacteria or bacterium that has been irradiated (ultraviolet (UV), X-ray, electron beam or gamma radiation), heated, or chemically treated to inactivate, kill, while retaining its immunogenicity. In one embodiment, the inactivated bacteria disclosed herein are inactivated by treatment with an inactivating agent. Suitable inactivating agents include beta-propiolactone, binary or beta- or acetyl-ethyleneimine, glutaraldehyde, ozone, and Formalin (formaldehyde).

For inactivation by formalin or formaldehyde, formaldehyde is typically mixed with water and methyl alcohol to create formalin. The addition of methyl alcohol prevents degradation or cross reaction during the in activation process. One embodiment uses about 0.1 to 1% of a 37% solution of formaldehyde to inactivate the bacteria or bacterium. It is critical to adjust the amount of formalin to ensure that the material is inactivated but not so much that side effects from a high dosage occur.

A more preferred inactivation method is the use of Ethylenimine and related derivatives, such as binary ethylenimine (BEI) and acetylethylenimine, are examples of suitable chemical inactivating agents for use in inactivating the PED bacteria. Other chemical inactivating agents, e.g., beta-propiolactone, aldehydes (such as formaldehyde) and/or detergents (e.g., TWEEN® detergent, TRITON® X, or alkyl trimethylammonium salts) can also be used to inactivate the bacteria. The inactivation can be performed using standard methods known to those of skill in the art. Samples can be taken at periodic time intervals and assayed for residual live bacteria. Monitoring of cytopathic effect on an appropriate cell line and/or fluorescent staining with an appropriate specific monoclonal or polyclonal antibody can be used to detect the presence of residual live bacteria.

Inactivation with BEI can be accomplished by combining a stock BEI solution (e.g., a solution formed by adding 0.1-0.2 M 2-bromo-ethylamine hydrobromide to 0.1-0.2 N aqueous NaOH) with viral fluids to a final concentration of about 1-5 mM BEI. Inactivation is commonly performed by holding the BEI-bacteria mixture at 35-40° C. (e.g., 37° C.) with constant mixing for 24-72 hours. Bacteria inactivation can be halted by the addition of sodium thiosulfate solution to a final concentration in excess of the BEI concentration (e.g., addition of sodium thiosulfate at 17% of the volume of BEI to neutralize excess BEI) followed by mixing.

More particularly, the term “inactivated” in the context of a bacteria means that the bacteria is incapable of replication in vivo or in vitro and, respectively, the term “inactivated” in the context of a bacteria means that the bacteria is incapable of reproduction in vivo or in vitro. For example, the term “inactivated” may refer to a bacteria that has been propagated in vitro, e.g., in vitro, and has then deactivated using chemical or physical means so that it is no longer capable of replicating. In another example, the term “inactivated” may refer to bacteria that has been propagated, and then deactivated using chemical or physical means resulting in a suspension of the bacteria, fragments or components of the bacteria, such as resulting in a solution which may be used as a component of a vaccine.

The term “live vaccine” refers to a vaccine comprising a living, in particular, a living bacterial active component.

A “pharmaceutical composition” essentially consists of one or more ingredients capable of modifying physiological, e.g., immunological functions, of the organism it is administered to, or of organisms living in or on the organism. The term includes, but is not restricted to, antibiotics or antiparasitics, as well as other constituents commonly used to achieve certain other objectives such as, but not limited to, processing traits, sterility, stability, feasibility to administer the composition via enteral or parenteral routes such as oral, intranasal, intravenous, intramuscular, subcutaneous, intradermal, or other suitable route, tolerance after administration, or controlled release properties. One non-limiting example of such a pharmaceutical composition, solely given for demonstration purposes, could be prepared as follows: cell culture supernatant of an infected cell culture is mixed with a stabilizer (e.g., spermidine and/or bovine serum albumin (BSA)) and the mixture is subsequently lyophilized or dehydrated by other methods. Prior to vaccination, the mixture is then rehydrated in aqueous (e.g., saline, phosphate buffered saline (PBS)) or non-aqueous solutions (e.g., oil emulsion, aluminum-based adjuvant).

As used herein, “pharmaceutical- or veterinary-acceptable carrier” includes any and all solvents, dispersion media, coatings, adjuvants, stabilizing agents, diluents, preservatives, antibacterial and antifungal agents, isotonic agents, adsorption delaying agents, and the like. In some preferred embodiments, and especially those that include lyophilized immunogenic compositions, stabilizing agents for use in the present invention include stabilizers for lyophilization or freeze-drying.

In some embodiments, the immunogenic composition of the present invention contains an adjuvant. “Adjuvants” as used herein, can include aluminum hydroxide and aluminum phosphate, saponins e.g., Quil A, QS-21 (Cambridge Biotech Inc., Cambridge Mass.), GPI-0100 (Galenica Pharmaceuticals, Inc., Birmingham, Ala.), water-in-oil emulsion, oil-in-water emulsion, water-in-oil-in-water emulsion. The emulsion can be based in particular on light liquid paraffin oil (European Pharmacopea type); isoprenoid oil such as squalane or squalene; oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed. Stewart-Tull, D. E. S.), John Wiley and Sons, NY, pp 51-94 (1995) and Todd et al., Vaccine 15:564-570 (1997). Exemplary adjuvants are the SPT emulsion described on page 147 of “Vaccine Design, The Subunit and Adjuvant Approach” edited by M. Powell and M. Newman, Plenum Press, 1995, and the emulsion MF59 described on page 183 of this same book.

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name CARBOPOL® (BF Goodrich, Ohio, USA) are particularly appropriate. They are cross-linked with an allyl sucrose or with allyl pentaerythritol. Among then, there may be mentioned Carbopol 974P, 934P and 971P. Most preferred is the use of Carbopol 971P. Among the copolymers of maleic anhydride and alkenyl derivative, are the copolymers EMA (Monsanto), which are copolymers of maleic anhydride and ethylene. The dissolution of these polymers in water leads to an acid solution that will be neutralized, preferably to physiological pH, in order to give the adjuvant solution into which the immunogenic, immunological or vaccine composition itself will be incorporated.

Further suitable adjuvants include, but are not limited to, the RIBI adjuvant system (Ribi Inc.), Block co-polymer (CytRx, Atlanta Ga.), SAF-M (Chiron, Emeryville Calif.), monophosphoryl lipid A, Avridine lipid-amine adjuvant, heat-labile enterotoxin from E. coli (recombinant or otherwise), cholera toxin, IMS 1314 or muramyl dipeptide, or naturally occurring or recombinant cytokines or analogs thereof or stimulants of endogenous cytokine release, among many others.

It is expected that an adjuvant can be added in an amount of about 100 μg to about 10 mg per dose, preferably in an amount of about 100 μg to about 10 mg per dose, more preferably in an amount of about 500 μg to about 5 mg per dose, even more preferably in an amount of about 750 μg to about 2.5 mg per dose, and most preferably in an amount of about 1 mg per dose. Alternatively, the adjuvant may be at a concentration of about 0.01 to 50%, preferably at a concentration of about 2% to 30%, more preferably at a concentration of about 5% to 25%, still more preferably at a concentration of about 7% to 22%, and most preferably at a concentration of 10% to 20% by volume of the final product.

“Diluents” can include water, saline, dextrose, ethanol, glycerol, and the like. Isotonic agents can include sodium chloride, dextrose, mannitol, sorbitol, and lactose, among others. Stabilizers include albumin and alkali salts of ethylendiamintetracetic acid (EDTA), among others.

“Isolated” means altered “by the hand of man” from its natural state, i.e., if it occurs in nature, it has been changed or removed from its original environment, or both. For example, a polynucleotide or polypeptide naturally present in a living organism is not “isolated,” but the same polynucleotide or polypeptide separated from the coexisting materials of its natural state is “isolated”, as the term is employed herein.

“Attenuation” means reducing the virulence of a pathogen. In the present invention, an avirulent bacteria is one in which the virulence has been reduced so that it does not cause clinical signs of a Salmonella infection but is capable of inducing an immune response in the target mammal, but may also mean that the clinical signs are reduced in incidence or severity in animals infected with the inactivated or avirulent Salmonella in comparison with a “control group” of animals infected with non-avirulent, wild type Salmonella and not receiving the inactivated or avirulent bacteria. In this context, the term “reduce/reduced” means a reduction of at least 10%, preferably 25%, even more preferably 50%, still more preferably 60%, even more preferably 70%, still more preferably 80%, even more preferably 90% and most preferably of 100% as compared to the control group as defined above. Thus, an inactivated, avirulent and/or avirulent Salmonella isolate is one that suitable for incorporation into an immunogenic composition comprising an inactivated or an avirulent live Salmonella.

An “avirulent bacteria” is a viable (“live”) bacteria, in which the virulence of the infectious agent has been reduced, e.g., though passaging the bacteria in a specific cell line, or through genetic manipulation of the viral genome. The attenuation of the bacteria pertains to its virulence (pathogenicity), but does not necessarily affect the replicative capability of a bacteria. Avirulent bacteria may still be capable of replication. Thus, it may be a strain of a bacteria whose pathogenicity has been reduced so that it will initiate the immune response without causing the specific disease. In the context of the present invention, an avirulent bacteria may be a Salmonella whose pathogenicity has been abrogated or reduced by inactivating at least one gene or protein involved in virulence. In the present invention “attenuation” is synonymous with “avirulent”. In this context, the term “reduce/reduced” means a reduction in pathogenicity of at least 10%, preferably 25%, even more preferably 50%, still more preferably 60%, even more preferably 70%, still more preferably 80%, even more preferably 90% and most preferably of 100% as compared to a control group.

“Virulent” refers to the ability of a Salmonella isolate to cause disease associated with Salmonella. Virulence can be evaluated by observing disease progression in the animal. An example of a “virulent” isolate of Salmonella is that exemplified by the challenge strain, as described and used in the present invention.

“Avirulent” refers to isolates of Salmonella that are lacking in virulence. That is, avirulent strains, isolates, or constructs are non-pathogenic and are incapable of causing disease. As used herein the term “avirulent” is used synonymously with the term “non-virulent.”

As used herein the terms “strain” or “isolate” are used interchangeably.

The term “wild type Salmonella”, as used herein, is in particular directed to an infectious pathogenic Salmonella, which is particularly capable of infection in swine.

Herein, “effective dose” means, but is not limited to, an amount of antigen that elicits, or is able to elicit, an immune response that yields a reduction of clinical symptoms in an animal to which the antigen is administered.

As used herein, the term “effective amount” means, in the context of a composition, an amount of an immunogenic composition capable of inducing an immune response that reduces the incidence of or lessens the severity of infection or incident of disease in an animal. Particularly, an effective amount refers to a titer measured in tissue culture infectious dose 50 or plaque forming units per dose. Alternatively, in the context of a therapy, the term “effective amount” refers to the amount of a therapy which is sufficient to reduce or ameliorate the severity or duration of a disease or disorder, or one or more symptoms thereof, prevent the advancement of a disease or disorder, cause the regression of a disease or disorder, prevent the recurrence, development, onset, or progression of one or more symptoms associated with a disease or disorder, or enhance or improve the prophylaxis or treatment of another therapy or therapeutic agent.

The term “immunoreactive to Salmonella” as used herein means that the peptide or fragment elicits the immunological response against Salmonella.

The term “vector” as it is known in the art refers to a polynucleotide construct, typically a plasmid or a bacteria, used to transmit genetic material to a host cell. Vectors can be, for example, bacteria, plasmids, cosmids, or phage. A vector as used herein can be composed of either DNA or RNA. In some embodiments, a vector is composed of DNA. An “expression vector” is a vector that is capable of directing the expression of a protein encoded by one or more genes carried by the vector when it is present in the appropriate environment. Vectors are preferably capable of autonomous replication. Typically, an expression vector comprises a transcription promoter, a gene, and a transcription terminator. Gene expression is usually placed under the control of a promoter, and a gene is said to be “operably linked to” the promoter.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

Vectors and methods for making and/or using vectors (or recombinants) for expression can be by or analogous to the methods disclosed in: U.S. Pat. Nos. 4,603,112, 4,769,330, 5,174,993, 5,505,941, 5,338,683, 5,494,807, 4,722,848, 5,942,235, 5,364,773, 5,762,938, 5,770,212, 5,942,235, 382,425, PCT publications WO 94/16716, WO 96/39491, WO 95/30018; Paoletti, “Applications of pox bacteria vectors to vaccination: An update, ”PNAS USA 93: 11349-11353, October 1996; Moss, “Genetically engineered poxbacteriaes for recombinant gene expression, vaccination, and safety,” PNAS USA 93: 11341-11348, October 1996; Smith et al., U.S. Pat. No. 4,745,051 (recombinant baculobacteria); Richardson, C. D. (Editor), Methods in Molecular Biology 39, “Baculobacteria Expression Protocols” (1995 Humana Press Inc.); Smith et al., “Production of Human Beta Interferon in Insect Cells Infected with a Baculobacteria Expression Vector”, Molecular and Cellular Biology, December, 1983, Vol. 3, No. 12, p. 2156-2165; Pennock et al., “Strong and Regulated Expression of Escherichia coli B-Galactosidase in Infect Cells with a Baculovirus vector, ”Molecular and Cellular Biology March 1984, Vol. 4, No. 3, p. 406; EPAO 370 573; U.S. application Ser. No. 920,197, filed Oct. 16, 1986; EP Patent publication No. 265785; U.S. Pat. No. 4,769,331 (recombinant herpes virus); Roizman, “The function of herpes simplex virus genes: A primer for genetic engineering of novel vectors,” PNAS USA 93:11307-11312, October 1996; Andreansky et al., “The application of genetically engineered herpes simplex viruses to the treatment of experimental brain tumors,” PNAS USA 93: 11313-11318, October 1996; Robertson et al., “Epstein-Barr bacteria vectors for gene delivery to B lymphocytes”, PNAS USA 93: 11334-11340, October 1996; Frolov et al., “Alphavirus-based expression vectors: Strategies and applications,” PNAS USA 93: 11371-11377, October 1996; Kitson et al., J. Virol. 65, 3068-3075, 1991; U.S. Pat. Nos. 5,591,439, 5,552,143; WO 98/00166; allowed U.S. application Ser. Nos. 08/675,556, and 08/675,566 both filed Jul. 3, 1996 (recombinant adenovirus); Grunhaus et al., 1992, “Adenovirus as cloning vectors,” Seminars in Virology (Vol. 3) p. 237-52, 1993; Ballay et al. EMBO Journal, vol. 4, p. 3861-65, Graham, Tibtech 8, 85-87, April, 1990; Prevec et al., J. Gen Virol. 70, 42434; PCT WO 91/11525; Felgner et al. (1994), J. Biol. Chem. 269, 2550-2561, Science, 259: 1745-49, 1993; and McClements et al., “Immunization with DNA vaccines encoding glycoprotein D or glycoprotein B, alone or in combination, induces protective immunity in animal models of herpes simplex bacteria-2 disease”, PNAS USA 93: 11414-11420, October 1996; and U.S. Pat. Nos. 5,591,639, 5,589,466, and 5,580,859, as well as WO 90/11092, WO93/19183, WO94/21797, WO95/11307, WO95/20660; Tang et al., Nature, and Furth et al., Analytical Biochemistry, relating to DNA expression vectors, inter alia. See also WO 98/33510; Ju et al., Diabetologia, 41: 736-739, 1998 (lentiviral expression system); Sanford et al., U.S. Pat. No. 4,945,050; Fischbach et al. (Intracel); WO 90/01543; Robinson et al., Seminars in Immunology vol. 9, pp. 271-283 (1997), (DNA vector systems); Szoka et al., U.S. Pat. No. 4,394,448 (method of inserting DNA into living cells); McCormick et al., U.S. Pat. No. 5,677,178 (use of cytopathic bacteriaes); and U.S. Pat. No. 5,928,913 (vectors for gene delivery); as well as other documents cited herein.

As used herein, the terms “nucleic acid” and “polynucleotide” are interchangeable and refer to any nucleic acid. The terms “nucleic acid” and “polynucleotide” also specifically include nucleic acids composed of bases other than the five biologically occurring bases (adenine, guanine, thymine, cytosine and uracil).

The term “regulatory element” and “expression control element” are used interchangeably and refer to nucleic acid molecules that can influence the expression of an operably linked coding sequence in a particular host organism. These terms are used broadly to and cover all elements that promote or regulate transcription, including promoters, core elements required for basic interaction of RNA polymerase and transcription factors, upstream elements, enhancers, and response elements. Exemplary regulatory elements in prokaryotes include promoters, operator sequences and a ribosome binding sites. Regulatory elements that are used in eukaryotic cells can include, without limitation, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell.

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, bacteria, and mammals (including humans). A promoter can be inducible, repressible, and/or constitutive. Inducible promoters initiate increased levels of transcription from DNA under their control in response to some change in culture conditions, such as a change in temperature.

As used herein, the term “enhancer” refers to a type of regulatory element that can increase the efficiency of transcription, regardless of the distance or orientation of the enhancer relative to the start site of transcription.

Generation of a viral vector can be accomplished using any suitable genetic engineering techniques well known in the art, including, without limitation, the standard techniques of restriction endonuclease digestion, ligation, transformation, plasmid purification, and DNA sequencing, for example as described in Sambrook et al. (Molecular Cloning: A Laboratory Manual. Cold Spring Harbor Laboratory Press, N.Y. (1989)).

A viral vector can incorporate sequences from the genome of any known organism. The sequences can be incorporated in their native form or can be modified in any way to obtain a desired activity. For example, the sequences can comprise insertions, deletions or substitutions.

A viral vector can include coding regions for two or more proteins of interest. For example, the viral vector can include the coding region for a first protein of interest and the coding region for a second protein of interest. The first protein of interest and the second protein of interest can be the same or different. In some embodiments, the viral vector can include the coding region(s) for a third or a fourth protein of interest. The third and the fourth protein of interest can be the same or different. The total length of the two or more proteins of interest encoded by one viral vector can vary. For example, the total length of the two or more proteins can be at least about 400 amino acids, at least about 450 amino acids, at least about 500 amino acids, at least about 550 amino acids, at least about 600 amino acids, at least about 650 amino acids, at least about 700 amino acids, at least about 750 amino acids, at least about 800 amino acids, or longer.

Preferred viral vectors include baculovirus such as BaculoGold (BD Biosciences Pharmingen, San Diego, Calif.), in particular provided that the production cells are insect cells. Although the baculovirus expression system is preferred, it is understood by those of skill in the art that other expression systems will work for purposes of the present invention, namely the expression of E or Eᵣₙₛ into the supernatant of a cell culture. Such other expression systems may require the use of a signal sequence in order to cause E or Eᵣₙₛ expression into the media.

The term “clade” as it is known in the art refers to a group consisting of an ancestor and all its descendants, a single “branch” in a phylogenetic tree. The ancestor may be, as an example an individual, a population or a species. A genogroup can include multiple clades.

An “immune response” or “immunological response” means, but is not limited to, the development of a cellular and/or antibody-mediated immune response to the composition or vaccine of interest. Usually, an immune or immunological response includes, but is not limited to, one or more of the following effects: the production or activation of antibodies, B cells, helper T cells, suppressor T cells, and/or cytotoxic T cells, directed specifically to an antigen or antigens included in the composition or vaccine of interest. Preferably, the host will display either a therapeutic or a protective immunological (memory) response such that resistance to new infection will be enhanced and/or the clinical severity of the disease reduced. Such protection will be demonstrated by either a reduction in number of symptoms, severity of symptoms, or the lack of one or more of the symptoms associated with the infection of the pathogen, a delay in the of onset of viremia, reduced viral persistence, a reduction in the overall viral load and/or a reduction of viral excretion.

Herein, “specifically immunoreactive” refers to an immunoreactive protein or polypeptide that recognizes an antigen characteristic of salmonella or CT infection but does not react with an antigen characteristic of a strict challenge control.

“Protection against disease”, “protective immunity”, “functional immunity” and similar phrases, means a response against a disease or condition generated by administration of one or more therapeutic compositions of the invention, or a combination thereof, that results in fewer deleterious effects than would be expected in a non-immunized subject that has been exposed to disease or infection. That is, the severity of the deleterious effects of the infection are lessened in a vaccinated subject. Infection may be reduced, slowed, or possibly fully prevented, in a vaccinated subject. Herein, where complete prevention of infection is meant, it is specifically stated. If complete prevention is not stated then the term includes partial prevention.

Herein, “reduction of the incidence and/or severity of clinical signs” or “reduction of clinical symptoms” means, but is not limited to, reducing the number of infected subjects in a group, reducing or eliminating the number of subjects exhibiting clinical signs of infection, or reducing the severity of any clinical signs that are present in one or more subjects, in comparison to wild-type infection. For example, it should refer to any reduction of pathogen load, pathogen shedding, reduction in pathogen transmission, or reduction of any clinical sign symptomatic of CT. Preferably these clinical signs are reduced in one or more subjects receiving the therapeutic composition of the present invention by at least 10% in comparison to subjects not receiving the composition and that become infected. More preferably clinical signs are reduced in subjects receiving a composition of the present invention by at least 20%, preferably by at least 30%, more preferably by at least 40%, and even more preferably by at least 50%.

The term “increased protection” herein means, but is not limited to, a statistically significant reduction of one or more clinical symptoms which are associated with infection by an infectious agent in a vaccinated group of subjects vs. a non-vaccinated control group of subjects. The term “statistically significant reduction of clinical symptoms” means, but is not limited to, the frequency in the incidence of at least one clinical symptom in the vaccinated group of subjects is at least 10%, preferably 20%, more preferably 30%, even more preferably 50%, and even more preferably 70% lower than in the non-vaccinated control group after the challenge the infectious agent. Even more preferably, “statistically significant reduction of clinical symptoms” means a P-value <0.05 and/or a 95% Confidence Interval not including 0.

“Long-lasting protection” shall refer to “improved efficacy” that persists for at least 3 weeks, but more preferably at least 3 months, still more preferably at least 6 months. In the case of livestock, it is most preferred that the long lasting protection shall persist until the average age at which animals are marketed for meat.

“Safety” refers to the absence of adverse consequences in a vaccinated animal following vaccination, including but not limited to: potential reversion of a bacterium-based vaccine to virulence, clinically significant side effects such as persistent, systemic illness or unacceptable inflammation at the site of vaccine administration.

The terms “vaccination” or “vaccinating” or variants thereof, as used herein means, but is not limited to, a process which includes the administration of an immunogenic composition of the invention that, when administered to an animal, elicits, or is able to elicit—directly or indirectly—, an immune response in the animal against Salmonella.

“Mortality”, in the context of the present invention, refers to death caused by Salmonella infection, and includes the situation where the infection is so severe that an animal is euthanized to prevent suffering and provide a humane ending to its life.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration preferably for administration to a mammal, especially a pig. Associated with such container(s) can be a notice in the form prescribed by a governmental agency regulating the manufacture, use or sale of pharmaceuticals or biological products, which notice reflects approval by the agency of manufacture, use or sale for human administration.

Preferably, the Salmonella according to the invention is an inactivated or live avirulent Salmonella bacteria and/or an avirulent live culture.

Preferably, the mutation, as described herein, comprises or consists of one or more point mutations and/or one or more genomic deletions and/or one or more insertions.

According to a further embodiment, the present invention also relates to a vector that comprises any of such nucleic acid molecules as described herein. In other words, the present invention relates to a vector, that includes the coding sequence of any such Salmonella, or part thereof. Preferably, said vector is an expression vector, which allows the expression of any such Salmonella or part thereof. Vectors according to the invention are those which are suitable for the transfection or infection of bacterial, yeast or animal cells, in vitro or in vivo.

The present vaccines typically include inactivated or avirulent Salmonellae formulated with a pharmaceutically acceptable carrier. The pharmaceutical forms suitable for injectable use commonly include sterile aqueous solutions (where water soluble) or dispersions and sterile powders for the extemporaneous preparation of sterile injectable solutions or dispersion. The formulation should desirably be sterile and fluid to the extent that easy syringability exists. The dosage form should be stable under the conditions of manufacture and storage and typically is preserved against the contaminating action of microorganisms such as bacteria and fungi. The carrier can be a solvent or dispersion medium containing, for example, water, ethanol, polyol (for example, glycerol, propylene glycol, liquid polyethylene glycol, and the like), suitable mixtures thereof and vegetable oils. One possible carrier is a physiological salt solution. The proper fluidity of the solution can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabenes, chlorobutanol, phenol, sorbic acid, thimerosal (sodium ethylmercuri-thiosalicylate), deomycin, gentamicin and the like. In many cases it may be preferable to include isotonic agents, for example, sugars or sodium chloride. Prolonged absorption of the injectable compositions, if desired, can be brought about by the use in the compositions of agents delaying absorption, for example, aluminum monostearate and gelatin.

The volume of a single dose of the vaccine of this invention may vary but will be generally within the ranges commonly employed in conventional vaccines. The volume of a single dose is preferably between about 0.1 ml and about 3 ml, preferably between about 0.2 ml and about 1.5 ml, more preferably between about 0.2 ml and about 0.5 ml at the concentrations of conjugate and adjuvant noted above.

The vaccine compositions of the invention may be administered by any convenient means.

The subject to which the composition is administered is preferably an animal, including but not limited to cows, horses, sheep, pigs, poultry (e.g. chickens), goats, cats, dogs, hamsters, mice and rats, most preferably the mammal is a swine, more preferably, a pregnant sow, gilt or piglet.

The formulations of the invention comprise an effective immunizing amount of one or more immunogenic compositions and a physiologically acceptable vehicle. Vaccines comprise an effective immunizing amount of one or more immunogenic compositions and a physiologically acceptable vehicle. The formulation should suit the mode of administration.

The immunogenic composition, if desired, can also contain minor amounts of wetting or emulsifying agents, or pH buffering agents. The immunogenic composition can be a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

Preferred routes of administration include but are not limited to intranasal, oral, intradermal, and intramuscular. Administration orally, most preferably in a single dose, is desirable. Oral methods include, but are not limited to direct oral administration or oral gavage, and via drinking water, preferably through use of an automatic whole-barn dosing device. The most preferred oral dosing method is via drinking water via and an automatic whole-barn dosing device. The skilled artisan will recognize that compositions of the invention may also be administered in one, two or more doses, as well as, by other routes of administration. For example, such other routes include subcutaneously, intracutaneously, intravenously, intravascularly, intraarterially, intraperitnoeally, intrathecally, intratracheally, intracutaneously, intracardially, intralobally, intramedullarly, or intrapulmonarily. Depending on the desired duration and effectiveness of the treatment, the compositions according to the invention may be administered once or several times, also intermittently, for instance on a daily basis for several days, weeks or months and in different dosages.

Embodiments of the invention also include a method for protecting a piglet against diseases associated with salmonella, comprising administering to a pig, any of the killed or avirulent vaccines described herein. For example, the administered vaccine comprises two or more antigens of Salmonella.

Thus according to one aspect, the present invention relates to a method for reducing the percentage of salmonella infections in a herd of pigs comprising the step administering to pigs an effective amount of inactivated or avirulent live salmonella antigen or an immunogenic composition comprising Salmonella antigen, wherein the Salmonella antigen is an inactivated or avirulent Salmonella antigen.

In a preferred embodiment the Salmonella of the invention is a combination of an effective amount of an SC described in U.S. Pat. No. 5,436,001 and U.S. Pat. No. 5,580,557; and an attenuated ST isolate registered as 421/125 by IDT and derived from the wild type isolate S. Typhimurium 415.

The compounds described herein can be administered to a subject at therapeutically effective doses to treat Salmonella associated diseases. The dosage will depend upon the host receiving the vaccine as well as factors such as the size, weight, and age of the host.

Immunogenicity of a composition can be determined by monitoring the immune response of test subjects following immunization with the composition by use of any immunoassay known in the art. Generation of a humoral (antibody) response and/or cell-mediated immunity may be taken as an indication of an immune response. Test subjects may include animals such as pigs, mice, hamsters, dogs, cats, rabbits, cows, horses, sheep, and poultry (e.g. chickens, ducks, geese, and turkeys).

The immune response of the test subjects can be analyzed by various approaches such as: the reactivity of the resultant immune serum to the immunogenic conjugate, as assayed by known techniques, e.g., enzyme linked immunosorbent assay (ELISA), immunoblots, immunoprecipitations, etc.; or, by protection of immunized hosts from infection by the pathogen and/or attenuation of symptoms due to infection by the pathogen in immunized hosts as determined by any method known in the art, for assaying the levels of an infectious disease agent, e.g., the bacterial levels (for example, by culturing of a sample from the subject), or other technique known in the art. The levels of the infectious disease agent may also be determined by measuring the levels of the antigen against which the immunoglobulin was directed. A decrease in the levels of the infectious disease agent or an amelioration of the symptoms of the infectious disease indicates that the composition is effective.

The therapeutics of the invention can be tested in vitro for the desired therapeutic or prophylactic activity, prior to in vivo use in animals or humans. For example, in vitro assays that may be used to determine whether administration of a specific therapeutic is indicated include in vitro cell culture assays in which appropriate cells from a cell line or cells cultured from a subject having a particular disease or disorder are exposed to or otherwise administered a therapeutic, and the effect of the therapeutic on the cells is observed.

Alternatively, the therapeutic may be assayed by contacting the therapeutic to cells (either cultured from a subject or from a cultured cell line) that are susceptible to infection by the infectious disease agent but that are not infected with the infectious disease agent, exposing the cells to the infectious disease agent, and then determining whether the infection rate of cells contacted with the therapeutic was lower than the infection rate of cells not contacted with the therapeutic. Infection of cells with an infectious disease agent may be assayed by any method known in the art.

In addition, the therapeutic can be assessed by measuring the level of the molecule against which the antibody is directed in the animal model or human subject at suitable time intervals before, during, or after therapy. Any change or absence of change in the amount of the molecule can be identified and correlated with the effect of the treatment on the subject. The level of the molecule can be determined by any method known in the art.

After vaccination of an animal to a Salmonella vaccine or immunogenic composition using the methods and compositions of the present invention, any binding assay known in the art can be used to assess the binding between the resulting antibody and the particular molecule. These assays may also be performed to select antibodies that exhibit a higher affinity or specificity for the particular antigen.

The invention extends to Salmonella isolates which are derived from the strains through propagation or multiplication in an identical or divergent form, in particular descendants which possess the essential characteristics of the deposited strains. Upon continued propagation, the strains may acquire mutations most of which will not alter the properties of these strains significantly.

The Salmonella isolates of the present invention are suitable for vaccines of the invention can be grown and harvested by methods known in the art, e.g., by propagating in suitable host cells.

In particular, the vaccine, as mentioned herein, is a live vaccine and/or a modified live vaccine-avirulent vaccine. The strains of the Salmonella according to the invention can be grown and harvested by methods known in the art, e.g. by propagating in suitable cells Modified live vaccines (MLV) are typically formulated to allow administration of 10¹ to 10⁷ bacterial particles per dose, preferably 10³ to 10⁶ particles per dose, and more preferably 10⁴ to 10⁶ particles per dose (4.0-6.0 log₁₀ TCID₅₀).

Antibodies, or binding portions thereof, resulting from the use of Salmonella peptides of the present invention are useful for detecting in a sample the presence of Salmonella. This detection method comprises the steps of providing an isolated antibody or binding portion thereof raised against an Salmonella peptide of the invention, adding to the isolated antibody or binding portion thereof a sample suspected of containing a quantity of Salmonella and detecting the presence of a complex comprising the isolated antibody or binding portion thereof bound to Salmonella.

The antibodies or binding portions thereof of the present invention are also useful for detecting in a sample the presence of a Salmonella peptide. This detection method comprises the steps of providing an isolated antibody or binding portion thereof raised against a Salmonella peptide, adding to the isolated antibody or binding portion thereof a sample suspected of containing a quantity of the Salmonella peptide, and detecting the presence of a complex comprising the isolated antibody or binding portion thereof bound to the Salmonella peptide.

Immunoglobulins, particularly antibodies, (and functionally active fragments thereof) that bind a specific molecule that is a member of a binding pair may be used as diagnostics and prognostics, as described herein. In various embodiments, the present invention provides the measurement of a member of the binding pair, and the uses of such measurements in clinical applications. The immunoglobulins in the present invention may be used, for example, in the detection of an antigen in a biological sample whereby subjects may be tested for aberrant levels of the molecule to which the immunoglobulin binds, and/or for the presence of abnormal forms of such molecules. By “aberrant levels” is meant increased or decreased relative to that present, or a standard level representing that present, in an analogous sample from a portion of the body or from a subject not having the disease. The antibodies of this invention may also be included as a reagent in a kit for use in a diagnostic or prognostic technique.

In one aspect, an antibody of the invention that immunospecifically binds to a salmonella peptide may be used to diagnose, prognose or screen for a Salmonella infection.

In another aspect, the invention provides a method of diagnosing or screening for the presence of a Salmonella infection or immunity thereto, comprising measuring in a subject the level of immunospecific binding of an antibody to a sample derived from the subject, in which the antibody immunospecifically binds a Salmonella peptide in which an increase in the level of said immunospecific binding, relative to the level of said immunospecific binding in an analogous sample from a subject not having the infectious disease agent, indicates the presence of Salmonella.

Examples of suitable assays to detect the presence of Salmonella peptides or antagonists thereof include but are not limited to ELISA, radioimmunoassay, gel-diffusion precipitation reaction assay, immunodiffusion assay, agglutination assay, fluorescent immunoassay, protein A immunoassay, or immunoelectrophoresis assay.

Immunoassays for the particular molecule will typically comprise incubating a sample, such as a biological fluid, a tissue extract, freshly harvested cells, or lysates of cultured cells, in the presence of a detectably labeled antibody and detecting the bound antibody by any of a number of techniques well-known in the art.

The binding activity of a given antibody may be determined according to well-known methods. Those skilled in the art will be able to determine operative and optimal assay conditions for each determination by employing routine experimentation.

An additional aspect of the present invention relates to diagnostic kits for the detection or measurement of Salmonella. Kits for diagnostic use are provided, that comprise in one or more containers an anti-salmonella peptide antibody, and, optionally, a labeled binding partner to the antibody. Alternatively, the anti-salmonella peptide antibody can be labeled (with a detectable marker, e.g., a chemiluminescent, enzymatic, fluorescent, or radioactive moiety). Accordingly, the present invention provides a diagnostic kit comprising, an anti-Salmonella peptide antibody and a control immunoglobulin. In a specific embodiment, one of the foregoing compounds of the container can be detectably labeled. A kit can optionally further comprise in a container predetermined amounts of a Salmonella peptide recognized by the antibody of the kit, for use as a standard or control.

Yet another embodiment of the invention includes a kit for vaccinating a pregnant sow or gilt against diseases associated with Salmonella comprising: a dispenser capable of administering a vaccine to a pregnant sow or gilt; and a Salmonella vaccine as described herein.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

The purpose of this study was to evaluate the fecal shedding of Salmonella Typhimurium challenge in vaccinated pigs. Pigs were administered Salmonella Choleraesuis-Typhimurium Vaccine, Avirulent Live Culture in the drinking water at approximately 14 days of age and challenged four weeks later. Demonstration of prevention of fecal shedding or reduction in duration of fecal shedding in minimum age pigs vaccinated with an immunizing dose of the vaccine of the present invention is the goal.

The materials for the challenge preparation were prepared as follows:

Glycerol stock of Salmonella Typhimurium was removed from the freezer, thawed and 200 ul was added to two 1 L beveled flasks containing 200 mL of growth media, Porcine BHI with glucose. The flasks were incubated overnight at 37° C. shaking at 200 rpm. From the overnight cultures, 10% was added to two flasks containing growth media. The inoculated flasks which were incubated at 37° C. shaking at 250 rpm for four hours. The cultures were centrifuged for concentration at 6000×G for 15 minutes using the JLA-8.1 rotor. After centrifugation, the supernatant was decanted and the pellets were resuspended in 200 mL of spent media. Sterile glycerol was added to the pooled culture (30 mL). The culture was dispensed to vaccine bottles (2×100 mL and 10×2 mL cryovials) and frozen at <−60° C. One 2 mL cryovial was removed from the freezer and titrated. The titration was done on the undiluted culture and a dilution of 0.25 mL into 120 mL of sterile diluent. 1:10 serial dilutions were made in PBS and 100 ul was plated to tryptic soy agar plates, in duplicate. The colony counts from the TSA plates were used determine the Salmonella Typhimurium count; counts were calculated by averaging the plate counts with 30 to 300 colonies per plate. The average titer of ST was 2.30×10¹⁰ cfu/mL and the average titer of ST was 4.60×10¹⁰ cfu/2 mL ds. When diluted 1:480, the average titer of ST was 1.2×10⁷ cfu/mL and the average titer of ST was 2.4o×10⁷ cfu/2 mL ds.

One frozen 2 mL vial of Salmonella Typhimurium was completely thawed and gently mixed. 0.5 mL of the culture was added to the 120 mL of sterile diluent in the PETG container and swirled to mix. 1.5 mL was removed and transferred to each of the two empty cryovials. Both vials were frozen at <−60 C. The remaining contents were divided to the two vaccine bottles, capped and administered at 2 mL per pig.

One of the retention samples was thawed and titrated in a similar manner as above. 1:10 serial dilutions were made in PBS and 100 ul was plated to tryptic soy agar plates, in triplicate. The colony counts from the TSA plates were used determine the Salmonella Typhimurium count; counts were calculated by averaging the plate counts with 30 to 300 colonies per plate. The retention samples were stored at <−60° C. The Material Average Titer of ST was 6.63×10⁷ cfu/mL and 1.33×10⁸ cfu/2 mL ds.

The Salmonella Choleraesuis-Salmonella Typhimurium Vaccine was rehydrated with 100 mL of diluent. 100 ul of this reconstituted formulation was struck for purity evaluation on a BAP, incubated aerobically and anaerobically for three days at 37° C. A retention sample was taken for potency performed by the colony forming unit titration method utilizing triplicate plates and a subtractive count for the quantification of live Salmonella Choleraesuis-Typhimurium ALC combination vaccine. 1:10 serial dilutions were made in PBS and 100 ul was plated to TSA and Ade−/His− agar plates, in triplicate. The counts from the TSA plates were used determine the total Salmonella count, representing both isolates. The Ade−/His− plate count represented the SC count. The SC count was subtracted from the total to determine the ST count.

In this study, the vaccine was administered once through the drinking water to two-week-old pigs on Day 0 (D0), and pigs were challenged with virulent ST four weeks later.

TABLE 1
SCHEDULE OF EVENTS

| Study Day | Study Event |
|---|---|
| D −1 | Animal Health Examination |
| | Feed, Blood & Fecal Sample Collection |
| | Body Weights |
| D 0 (Pre- | Daily Assessment |
| vaccination) | |
| D 0 | Vaccination |
| D 1-D 112 | Daily Assessment |
| D 2-D 4 | Fecal Sample Collection |
| D 27 | Pigs moved to Challenge Rooms |
| D 27 | Feed, Blood & Fecal Sample Collection |
| | Body Weights |
| D 28 | Pre-Challenge Fecal Collection |
| | Challenge |
| D 29-D 42 | Daily Fecal Collection |
| Between D 43 | Fecal Collection (three non-consecutive |
| and D 111 | collections per week) |
| D 112 | Feed, Blood & Fecal Sample Collection |
| | Body Weights |
| D 112 | Off-test/Disposition |

TABLE 2
VACCINE AND PLACEBO FORMULATION

| Treatment | Description* |
|---|---|
| Placebo | Lyophilized Control Product containing media and stabilizer |
| | with the same quantity and component concentration as the |
| | Vaccine without SC/ST ALC organisms. |
| Vaccine | Lyophilized vaccine containing Salmonella Choleraesuis, |
| | strain 54 and Salmonella Typhimurium, strain 421/125 will be |
| | diluted to target 6.54 × 10⁷ CFU and 4.54 × 10⁸ CFU per |
| | dose, respectively. |

*ENTERISOL ® Ileitis, USDA Product Code 10L1.01, will also be administered to all pigs in both treatment groups as per label directions, concurrently in the drinking water.

On D-1, at approximately the same time of day as the scheduled vaccination, the water usage of the pigs' pens was measured to obtain a baseline for water intake. After five hours, the volume of water solution drawn from each pen's container was recorded. This pre-measured volume in each pen was used for the vaccine and drinking water solution on the day of vaccination per pen, respectively.

The morning of D0 vaccination, the lyophilized vaccine was rehydrated and diluted appropriately for the treatments. A stock solution of water was stabilized with RELOAD PACK® DT (Boehringer Ingelheim Vetmedica, Inc.) per label directions. The appropriate number of 2-mL Vaccine or Placebo doses and vaccine were added to the waterer reservoirs. The stabilized water solution was then added to the waterer reservoirs until the total volume (solution plus doses) equaled the premeasured five-hour volume of drinking water for each pen. A 4- to 5-mL sample from the final treatment preparation was collected to represent the vaccination material. This retention sample was titrated at the time of vaccination; remaining sample was labeled and then frozen to −60° C. or colder.

Treatments were administered by a Dose Administrator (an individual not responsible for collection of other data variables during the challenge phase). The final treatment preparation was supplied by ad libitum group access through a conventional nipple/cup waterer such as “Aqua Chief Wean/Finish Cup” or comparable commonly available device mounted to the side of the pen. After six hours, any remaining treatment was measured, recorded and returned to the waterer. Pens were checked every one to two hours until all treatment was consumed and documented. Once treatment was consumed in a room, fresh drinking water was supplied ad libitum. The pens had one waterer per pen of six pigs (three pigs from each of two litters); four pens per treatment.

TABLE 3
SALMONELLA TYPHIMURIUM CHALLENGE MATERIAL

| | |
|---|---|
| Name/number of isolate | Salmonella Typhimurium, Universal Killer Strain (ST-UK) |
| Titer | 6.63 × 10⁷ cfu/mL |
| Storage conditions | Challenge material was stored on ice packs after preparation. |
| Challenge Material | Isolate was obtained from NADC; see Curtiss, RI, et al. 1991. |
| | “Colonization control of human bacterial enteropathogens in |
| | poultry.” Nonrecombinant and recombinant avirulent Salmonella |
| | live vaccines for poultry. Academic Press, Inc., San Diego, CA p. |
| | 169-198 for description. Seed culture was grown from the ST |
| | challenge culture stock in static BHI broth media at 37° C. overnight, |
| | then 1% of the static culture was transferred to pre-warmed BHI |
| | media, incubated with agitation after 4-6 hours of incubation, |
| | harvested and frozen with glycerol. On the day of challenge the |
| | frozen material was completely thawed in a 32-37° C. water bath and |
| | diluted to the target dose. |
| Vial size | 25-100 mL sterile vaccine vials - suitable for withdrawal with |
| | syringe |
| Dose | 1.33 × ≧10⁸ cfu/2 mLdose |
| Administration route | Intranasally drop-wise from a syringe without a needle |

On D28, all pigs received 2 mL of challenge material intranasally. The investigator instilled 1 mL of challenge material into each nostril of each pig during inhalation.

TABLE 4
STUDY DESIGN

| Treatment | | | | | Fecal | Off-test/ |
|---|---|---|---|---|---|---|
| Group | Product | Pigs | Treatment | Challenge | Samples | Disposition |
| T01 | Placebo | 24 | D0 | D28 | Per | D112 |
| T02 | Vaccine | 24 | Drinking | 2 mL IN | collection | |
| | | | Water | *1.33 × 10⁸ | schedule | |
| | | | | cfu/dose | | |

*Dose targeted to induce shedding but not clinical disease (~8 logs/dose).

Each pig will be an experimental unit.

Twenty-four experimental units per treatment group and moderate variability (0.5 on logit scale) allowed the study design to have approximately 80% power to detect a Prevented Fraction significantly different from 0 (95% CI does not include 0) when 90% of the placebo and 45% of the vaccine experimental units shed bacteria post-challenge.

Randomization was conducted using a random number generator in SAS® version 9.2 (or later). Eight litters with six pigs were utilized for the study. Pigs within litter were randomly assigned to treatments such that three pigs within each litter were assigned to each of the treatment groups, T01 and T02.

Pigs in the T01 and T02 treatment groups were housed in separate rooms during the vaccination phase. Each vaccination phase room consisted of four pens with six pigs per pen and littermates in the same treatment group housed in the same pen.

Prior to challenge, pigs were moved to one of two rooms, where each room contained twenty-four pens. Pigs were housed individually, with littermates randomized to a block of six pens in close spatial proximity.

Throughout the study, any personnel involved in collecting data or performing laboratory assays/analyses were masked to treatment allocation (i.e., were blinded as to which treatment a group has received). Additionally, the aforementioned personnel were also masked to group membership (i.e., were blinded to group in which all pigs are assigned). Grouping of study animals was conducted via a blinded allotment/randomization discussed above. Treatments were administered by an individual not involved with any data collection and/or analyses in the study.

TABLE 5
ANIMAL INFORMATION

| Specifications | Requirements |
|---|---|
| Species & Breed | Porcine, commercial mixed breed |
| Age & Sex | 14-17 days of age on D 0, females and intact males |
| Identification | Ear tag (uniquely numbered) |
| Conditioning | Pigs originated from a herd without a history of clinical salmonellosis |
| | and without Salmonella vaccination. Fecal samples were collected from |
| | source sows and pigs and were cultured for Salmonella prior to purchase |
| | to determine active shedding. |
| Physiological status | All pigs were healthy at the time of vaccination as determined by |
| | observation. |

Only pigs that met the specifications of Table 5 were included in the study. Prior to the start of the study, a veterinarian conducted a Health Examination and only allowed healthy pigs to be included in the study (healthy and free of any medical conditions that would interfere with assessment of salmonellosis, such as dyspnea, abnormal fecal consistency or diarrhea, abnormal lethargy or dehydration).

After the start of the study, pigs were excluded only in the case of injury, illness (other than challenge-related) or death that interfered with the outcome of the study. At any time post-challenge, if cause of death was determined to be not challenge-related, the data will be excluded from the results.

A venous blood sample was collected from pigs by the study investigator or designee prior to vaccination (D-1), prior to challenge (D27) and prior to off-test/disposition. Approximately 4 to 10 mL of blood was collected from each pig with an appropriate-sized needle into an appropriate-sized Serum Separator Tube (SST). Blood collection was recorded. Blood in SSTs was allowed to clot at room temperature and centrifuged. Serum was harvested into aliquots using appropriate tubes by the Laboratory Scientist. Each aliquot was labeled and one aliquot from each sampling day was submitted for antibody testing using the IDEXX Swine Salmonella Ab Test.

Approximately 1 gram of feces or the maximum amount which was retrievable from the rectum was collected from all pigs prior to vaccination on D-1, following vaccination on D2, D3, D4, prior to challenge on D27, D28 and daily starting on D29 through D42. Thereafter, samples were collected three times per week on non-consecutive days through off-test/disposition. Sample collections were recorded and each fecal sample was labeled and frozen at −60° C. or colder on the day of collection. The samples were thawed and cultured for Salmonella.

Samples were cultured by enrichment methods for Salmonella using methods outlined below which have a sensitivity of approximately 400 CFU/gram of feces by enrichment culture from fresh samples and 4000 CFU/gram from frozen samples. Samples were considered positive or negative for Salmonella Typhimurium (challenge isolate). The culture by enrichment was adapted from, “Microbiological” Methods for Monitoring the Environment”, Environmental Monitoring and Support Laboratory, Office of Research and Development, US Environmental Protection Agency, Cincinnati, Ohio (1978); “Clinical Veterinary Microbiology”, Quinn, Carter, Market, Carter (1994); and “Culture Methods differ on the isolation of Salmonella enterica serotypes from naturally contaminated swine fecal samples”, M. Rostagno, et al. J Vet Diagn Invest 17:80-83 (2005).

Specifically, post-challenge fecal samples were inoculated into RV broth, incubated for 24 hours at 42° C., after incubation, one mL is transferred to 10 mL of RV broth and incubated for 24 hours at 42° C. After incubation, 100 uL is planted directly on xylose-lysine-deoxycholate (XLD) plate media and streaked for isolation. The plates were incubated at 37° C. for 18-24 hours. Then the plates were read for black colonies. Selected isolated black colonies are transferred to blood agar plates and serotyped Poly Al-Vi Salmonella antiserum and Salmonella specific O-group antiserum: Group B for ST.

Body weight was monitored during the study. The Study Investigator weighed pigs prior to vaccination (D-1), prior to challenge (D27) and prior to off-test/disposition using a calibrated scale. Body weights were documented. Any pig that died before its scheduled off-test/disposition will be weighed on the day it is found dead or euthanized.

For a valid study, the T01 pigs must remain healthy and fecal culture negative for vaccine strains prior to challenge. If a concurrent disease impacts the outcome of the study, the study may be considered invalid.

Fecal shedding presence and/or duration of Salmonella were determined using enrichment culture isolation methods from fecal samples collected daily. Detection of Salmonella Typhimurium (challenge isolate) will be considered positive; samples without detection of Salmonella shall be considered negative. A pig will be considered positive for shedding if the challenge isolate is detected in one or more post-challenge samples.

The body weights of each treatment group for the vaccination and challenge phases were evaluated. The average daily weight gain (ADWG) was also assessed.

Prevention of shedding of Salmonella was determined if a pig was enrichment culture negative. For reduction in duration of Salmonella shedding, the duration will be determined by the number of days from first positive to the last day positive, inclusive of the first and last day.

All data from the study was imported into SAS® version 9.2 (or later) for management and analysis. Data listings and summary statistics by treatment group, including frequency distributions, were generated. Prevented fractions (PF) and/or mitigated fractions (MF), along with 95% confidence limits, were used to assess study parameters. See Siev, D. 2005. “An estimator of intervention effect on disease severity,” Journal of Modern Applied Statistical Methods. Vol. 4, No. 2, pp 500-508, hereby incorporated by reference, for a comprehensive presentation of the MF.

For parameters which were assessed via the PF, a generalized linear mixed model (GLMM) which includes a fixed effect for treatment and a random effect for litter was used. The GLMM used a binomial distribution with a logit link function. If convergence of the maximization algorithm does not occur, the random effect may be removed from the model. The PF and associated confidence interval were calculated using the parameter and variance/covariance estimates from the GLMM analysis, where the delta method was used in determining the confidence interval. If 100% or 0% of the experimental units for either group is positive, the Two-One Sided Scores Test in Proc StatXact will be used to estimate the PF and 95% confidence interval.

Should sufficient experimental units in the T02 group shed bacteria to warrant an analysis of the duration of fecal shedding, the MF will be calculated and 95% confidence interval estimated using bootstrapping methods, stratifying by litter.

Weight gain was analyzed using repeated measures analysis of body weights. The model included treatment, time and treatment by time as fixed effects and included litter as a random effect. Covariance structure will be unstructured. All hypothesis testing was conducted using an α-level of 0.05.

TABLE 6
STUDY EVALUATION PARAMETERS

| | | | | Summary/ |
|---|---|---|---|---|
| | Data | Type of | Individual | Analysis by |
| Parameter | Form | Variable | Analysis | treatment |
| ST Fecal | Lab | Qualitative | Prevention of | FD by day |
| Shedding | Data | 0 = negative | Shedding Claim: | FD by if |
| | | 1 = positive | Pig is positive if ≧ | ever |
| | | | 1 post-challenge | positive |
| | | | sample is | |
| | | | positive, | PF vs T01 |
| | | | otherwise | |
| | | | negative | |
| ST Fecal | Lab | Qualitative | Reduction in | Descriptive |
| Shedding | Data | 0 = negative | Duration of | stats |
| Duration | | 1 = positive | Shedding Claim: | MF vs T01 |
| | | | Number of days | |
| | | | from first positive | |
| | | | to the last day | |
| | | | positive, | |
| | | | inclusive of the | |
| | | | first and last day. | |
| Weight | Body | Body weights: | Body weight at | Descriptive |
| Gain | Weight | D −1 | each time point | Stats |
| | Record | D 27 | | HT vs. T01 |
| | | disposition | Average daily | HT vs T01 |
| | | | weight gain for | |
| | | | challenge phase | |

FD = Frequency Distribution,
PF = Prevented Fraction,
MF = Mitigated Fraction

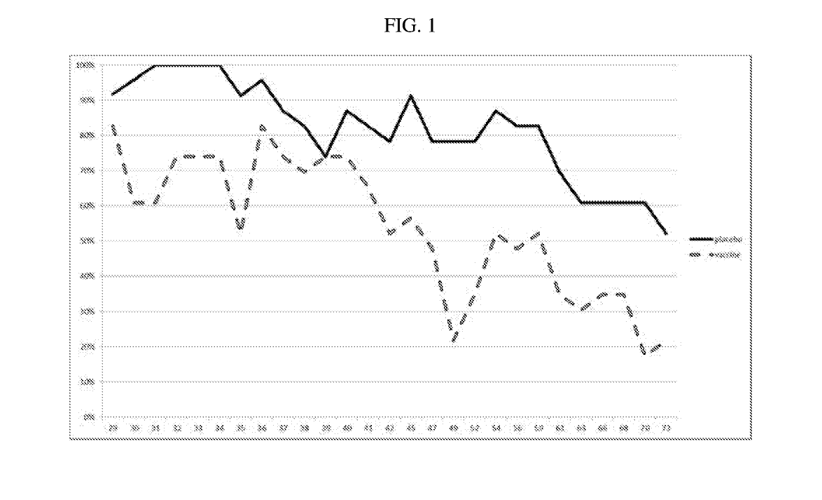

FIG. 1 shows the results of the shedding aspect of the study. Over the period represented, 45 days, the group of vaccinated pigs was consistently lower than the unvaccinated groups for fecal shedding of ST.

### Example 2

The objective of this vaccination-challenge study was to evaluate the fecal shedding of Salmonella Typhimurium challenge in vaccinated pigs. Pigs were administered Salmonella Choleraesuis-Typhimurium Vaccine, Avirulent Live Culture of the present invention in the drinking water at approximately 14 days of age and challenged four weeks later.

As Salmonella Typhimurium is an enteric pathogen in swine, shedding of the bacteria can occur intermittently upon infection. Shedding of such enteric pathogens in this manner is a common occurrence in the field and level of shedding depends on level of infection and persistence of the pathogen in infected herds. In order to more appropriately evaluate the shedding of Salmonella Typhimurium of pigs vaccinated with the Salmonella Choleraesuis-Typhimurium Vaccine, Avirulent Live Culture of the present invention compared to pigs that received placebo, adjustments to the Statistical Analyses were required.

A Prevented Fraction (PF) analysis was not conducted, as all study animals shed bacteria at least one day after challenge. In addition to duration of shedding, the number of positive samples post-challenge was analyzed by estimating the mitigated fraction (MF) and associated 95% confidence interval using bootstrapping methods, stratifying by litter. To characterize the proportion of animals shedding over time for each treatment group, a logistic model analysis of the proportion of animals shedding was conducted. The model included a fixed effect for treatment and a random effect for litter. The model was parameterized such that the midpoint estimates the number of days post-challenge where 50% of the animals were not shedding. A statistical test for a treatment effect on the number of days until 50% of the animals were not shedding was conducted at a 0.05 level of significance.

TABLE 7
SEQUENCE OF EVENTS

| Study Day | Date | Study Event |
|---|---|---|
| D −1 | 11 Jun. 2015 | Animal Health Examination |
| | | Blood & Fecal Sample |
| | | Collection Body Weights |
| D 0 (Pre-vaccination) | 12 Jun. 2015 | Daily Assessment |
| D 0 | 12 Jun. 2015 | Vaccination |
| D 1-D 112 | 13 Jun. 2015- | Daily Assessment |
| | 2 Oct. 2015 | |
| D 2-D 4 | 14 Jun. 2015- | Fecal Sample Collection |
| | 16 Jun. 2015 | |
| D 27 | 9 Jul. 2015 | Pigs moved to Challenge Rooms |
| D 27 | 9 Jul. 2015 | Blood & Fecal Sample |
| | | Collection Body Weights |
| D 28 | 10 Jul. 2015 | Pre-Challenge Fecal Collection |
| | | Challenge |
| D 29-D 42 | 11 Jul. 2015- | Daily Fecal Collection |
| | 24 Jul. 2015 | |
| Between D 43 and D 111 | 25 Jul. 2015- | Fecal Collection (three |
| | 1 Oct. 2015 | non-consecutive collections |
| | | per week) |
| D 112 | 2 Oct. 2015 | Feed, Blood & Fecal Sample |
| | | Collection Body Weights |
| D 112 | 2 Oct. 2015 | Off-test/Disposition |

TABLE 8
VACCINE AND CONTROL PRODUCTS FORMULATION

| Treatment | Laboratory I.D. | Description* |
|---|---|---|
| Placebo | Lot 441-193D | Lyophilized Control Product containing media and stabilizer with |
| | | the same quantity and component concentration as the Vaccine |
| | | without SC/ST ALC organisms. |
| Vaccine | Lot 441-193C | Lyophilized vaccine (#050312PD**) containing Salmonella |
| | | Choleraesuis, strain 54 and Salmonella Typhimurium, strain |
| | | 421/125 was diluted by BIVI-R&D, Ames IA to 1.07 × 10⁹ |
| | | CFU/mL and 9.35 × 10⁸ CFU/mL, respectively. Prepared according |
| | | to the Outline of Production for Product Code 19A2.00 at SC |
| | | passage of Master Seed + 10 and at ST passage of Master Seed + 11. |

*ENTERISOL ® Ileitis, USDA Product Code 10L1.01, was also administered to all pigs in both treatment groups as per label directions, concurrently in the drinking water.
**Same serial used to establish minimum immunizing dose, BIVI Studies 2012104 and 2013210.

On D-1, at approximately the same time of day as the scheduled vaccination, the water usage of the pens was measured to obtain a baseline for water intake. After five hours, the volume of water solution drawn from each pen's container was recorded as 900 mL, 1200 mL, 1200 mL and 1000 mL for Pens 1, 2, 3 and 4, respectively in room 5601 (T01), and 1300 mL, 700 mL, 1000 mL and 900 mL for Pens 1, 2, 3 and 4, respectively in room 5602 (T02). These pre-measured volumes were used for each respective pen to determine the vaccine concentration and drinking water solution.

The morning of D0, the lyophilized vaccine was rehydrated and labelled. This was then diluted appropriately for the treatments. Stock solution of water was stabilized with RELOAD PACK™ DT (Boehringer Ingelheim Vetmedica, Inc.) per label directions. The stabilized water solution was added to the waterer reservoirs until the total volume (solution plus doses) equaled the premeasured five-hour volume of drinking water for each pen. The appropriate number of 2-mL experimental vaccine or placebo doses and USDA Product Code 10L1.01 were added to the waterer reservoirs. A 5-mL sample from the final treatment preparations and placebo preparations were collected to represent the vaccination material. These retention samples were titrated at the time of vaccination; remaining samples were frozen to −60° C. or colder and stored. The waterer reservoirs were then transferred to the animal facility for vaccine/placebo administration.

TABLE 9
CHALLENGE MATERIAL

| | |
|---|---|
| Name/number of isolate | Salmonella Typhimurium, Universal Killer Strain (ST-UK) |
| Titer | 6.63 × 10⁷ CFU/mL (1.33 × 10⁸ CFU per 2 mL dose) |
| Storage conditions | Challenge material was stored on ice packs after preparation. |
| Challenge Material | Isolate was obtained from NADC; see Curtiss, RI et al 1991. |
| | “Colonization control of human bacterial enteropathogens in poultry.” |
| | Nonrecombinant and recombinant avirulent Salmonella live vaccines for |
| | poultry. Academic Press, Inc., San Diego, CA p. 169-198, hereby |
| | incorporated by reference, for description. Seed culture was grown from |
| | the ST challenge culture stock in static BHI broth media at 37° C. |
| | overnight, then 1% of the static culture was transferred to pre-warmed |
| | BHI media, incubated with agitation after 4-6 hours of incubation, |
| | harvested and frozen with glycerol. On the day of challenge the frozen |
| | material was completely thawed in a 32-37° C. water bath and diluted to the |
| | target dose. |
| Vial size | 25-100 mL sterile vaccine vials - suitable for withdrawal with syringe |
| Manufacturer | Boehringer Ingelheim Vetmedica, Inc., Ames, IA |
| Dose | 1.33 × 10⁸ CFU/2-mL dose |
| Administration route | Intranasally |
| Testing | A retention sample from the challenge event that was frozen at the time of |
| | challenge was used. Titration of the sample was conducted in triplicate. |
| Safety Precautions: | Since ST is a zoonotic organism, all laboratory personnel and animal |
| | technicians that came in contact with the ST challenge material took |
| | appropriate precautions. Site-specific protocols were followed which |
| | included, but are not limited to, the use of the following Personal |
| | Protective Equipment (PPE) (e.g., disposable gloves, face shield or |
| | protective eyewear, disposable boots and coveralls). |

TABLE 10
ANIMAL INFORMATION

| Specifications | Requirements |
|---|---|
| Species & Breed | Porcine, commercial mixed breed |
| Age & Sex | 14-17 days of age on D 0, females and intact males |
| Weight Range | 3.4 kg to 7.0 kg on D −1 |
| Source | Rock Creek, Nebraska City NE |
| Ownership | Midwest Veterinary Services, Inc. |
| Number | 48 |
| Identification | Par tag (uniquely numbered) |
| Conditioning | Pigs originated from a herd without a history of clinical salmonellosis and |
| | without Salmonella vaccination. Fecal samples were collected from source sows |
| | and pigs and cultured for Salmonella prior to purchase to determine active |
| | shedding. |
| Physiological status | All pigs were healthy at the time of vaccination as determined by observation. |

Only pigs that met the specifications outlined above were included in the study. Prior to the start of the study, a veterinarian conducted a Health Examination and only allowed healthy pigs to be included in the study (healthy and free of any medical conditions that would interfere with assessment of salmonellosis, such as dyspnea, abnormal fecal consistency or diarrhea, abnormal lethargy or dehydration). All pigs that arrived from the source herd were deemed healthy for study inclusion.

Two pigs were removed from the study: One T02 pig was removed during the vaccination phase and one T01 pig was removed during the challenge phase. Pig #42 (T02) was humanely euthanized on D20 (2 Jul. 2015) with lameness of the right front leg due to an abscess involving connective tissue in the region of the right humeroradial joint. Necropsy revealed a well-encapsulated abscess of muscle and other connective tissue around the joint that was likely a result of a bruise or other injury. Pig #27 (T01) was found dead on D31 from suspected septicemia due to Salmonella infection. Necropsy revealed fibrinous peritonitis, watery diarrhea, an enlarged spleen and moderate autolysis. A fecal sample was collected at necropsy and confirmed Salmonella positive. Prior to being humanely euthanized, Pig #42 (T02) was treated on D12 with 0.5 mL of EXCEDE® IM (ceftiofur crystalline free acid, Zoetis) in left neck and 0.1 mL FLU-NIX™ D IM (flunixin meglumine, Agri Labortories, Ltd.) left neck for lameness and for lameness in the right front leg and on D130.1 mL FLU-NIX™D IM in left neck for lameness. No other treatments other than those indicated above were administered to any animal during the study.

All animals were held in MVS Biosafety Level 2 isolation facilities and personnel at this facility followed local site procedures. Animal technicians responsible for feeding and managing pigs followed local site biosecurity procedures to prevent unintended exposure of SC/ST ALC or virulent ST challenge to other susceptible species, including humans.

Feeds used in this study were commercially obtained and appropriate for the stage and weight of the pigs. All feed used was non-medicated and contained no antibiotics. Feed was provided ad libitum. A feed retention sample was collected on D112 and will be retained in storage at 20° C. until project conclusion.

TABLE 11
METHODS

| | | | | | | Fecal | Off-test/ |
|---|---|---|---|---|---|---|---|
| Group | #V | Treatment* | CFU/mL | #C | Challenge | Samples | Disposition |
| T01 | 24 | D0 | None | 24 | D28 | Per | D112 |
| T02 | 24 | Drinking | SC: 1.07 × 10⁹ | 23 | IN** | collection | |
| | | Water | ST: 9.35 × 10⁸ | | | schedule | |

#V = number of pigs vaccinated;
#C = number of pigs challenged;
SC = Salmonella Choleraesuis;
ST = Salmonella Typhimurium;
CFU = colony forming units
*Pigs also received USDA Product Code 10L1.00 as per label directions concurrently in the drinking water.
**Dose was targeted to induce shedding but not clinical disease (titer was 1.33 × 10⁸ CFU/2 mL dose).

A venous blood sample was collected from pigs by the study investigator or designee prior to vaccination (D-1), prior to challenge (D27) and prior to off-test/disposition (D112). Approximately 4 to 10 mL of blood was collected from each pig with an appropriate-sized needle into an appropriate-sized Serum Separator Tube (SST). Blood in SSTs was allowed to clot at room temperature, centrifuged and delivered on ice packs to BIVI R&D-Ames. Serum was harvested into aliquots using appropriate tubes by the Laboratory Scientist or designee. Each aliquot was labeled with the pig's ID number, the study number, the study day of collection and the sample type. Retention serum samples have been stored at −10° C. or colder.

Approximately 1 gram of feces or the maximum amount which was retrievable from the rectum was collected from all pigs prior to vaccination on D-1, following vaccination on D2, D3, D4, prior to challenge (on D27 and D28) and daily starting on D29 through D42. Thereafter, samples were collected three times per week on non-consecutive days through off-test/disposition (D112). Each fecal sample was labeled with the animal's ID number, the study number, the study day of collection and the sample type. Fecal samples were frozen at −60° C. or colder on the day of collection. The samples were thawed and cultured for Salmonella.

Samples were cultured by enrichment methods for Salmonella using methods outlined above in Example 1, which has a sensitivity of approximately 4000 CFU/gram of feces by enrichment culture from frozen samples. Samples were considered positive or negative for group B Salmonella, within which is Typhimurium (challenge isolate). A subset of recovered group B isolates before and after the day of challenge were further characterized by serotyping for speciation at NVSL.

Beginning on D-3 and continuing throughout the study, all pigs were observed daily for general health, and observations were recorded. Prior to the start of the study, the Study Investigator's designee conducted a health exam and all pigs received were found to be healthy and were included in the study.

Body weight was monitored during the study. Pigs were weighed prior to vaccination (D-1), prior to challenge (D27) and prior to off-test/disposition (D112) using a calibrated scale in kilograms. Any pig that died before its scheduled off-test/disposition was weighed on the day it was found dead or euthanized. Any pig that died or required euthanasia for humane reasons after challenge was necropsied to determine the cause of death.

For a valid study, the T01 pigs had to remain healthy and fecal culture negative for vaccine strains prior to challenge. Fecal shedding presence and/or duration of Salmonella was determined using enrichment culture isolation methods from fecal samples collected throughout the study. Detection of group B Salmonella were considered positive; samples without detection of Salmonella were considered negative; results were not quantified. The body weights of each treatment group for the vaccination and challenge phases were evaluated. The average daily weight gain (ADWG) was also assessed.

Prevention of shedding of Salmonella was determined if a pig was enrichment culture negative. For reduction in duration of Salmonella shedding, the duration was determined by the number of days from first positive to the last day positive, inclusive of the first and last day. The statistical methods, including randomization and statistical analyses are the same as described in Example 1 above. The details are provided in Table 12.

TABLE 12
STUDY PARAMETERS

| | Data | Type of | | Summary/Analysis |
|---|---|---|---|---|
| Parameter | Form | Variable | Individual Analysis | by treatment |
| Fecal | Lab | Qualitative | Assessment of daily results | FD by day |
| Shedding | Data | 0 = negative | measured by group B Salmonella | PF by day |
| | | 1 = positive | | |
| Fecal | Lab | Qualitative | 1. Duration of shedding | Descriptive stats |
| Shedding | Data | 0 = negative | 2. Number of positive samples | MF vs T01 |
| Duration | | 1 = positive | Number of days when 50% of | HT vs. T01 |
| | | | animals are not shedding group B | |
| | | | Salmonella | |
| Weight | Body | Body weights | Body weight at each time point | Descriptive Stats |
| Gain | Weight | D −1 | | HT vs. T01 |
| | Record | D 27 | Average daily weight gain for | HT vs T01 |
| | | D 112 | challenge phase | |

FD = Frequency Distribution,
PF = Prevented Fraction,
MF = Mitigated Fraction,
HT = Hypothesis Testing

Prior to vaccination, all pigs were fecal culture negative for Salmonella. Post-vaccination (D2-D4) all T01 (control) pigs remained fecal culture negative while 10/24 T02 (vaccinated) pigs shed ALC vaccine bacteria (either ST and/or SC). Prior to challenge (D27 and D28), all T01 (control) pigs remained fecal culture negative. For T02 (vaccinated) pigs, 13/23 were fecal culture positive on one day each with twelve further identified as Salmonella enterica ser Derby and one as Rough_O:fg:-

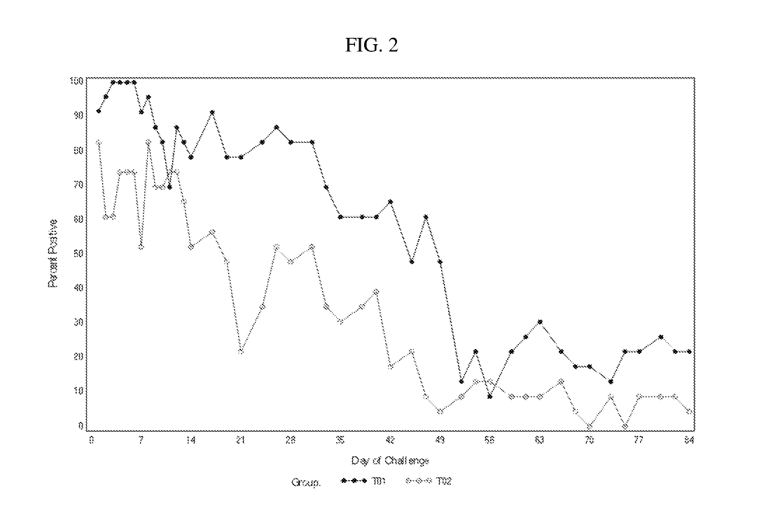

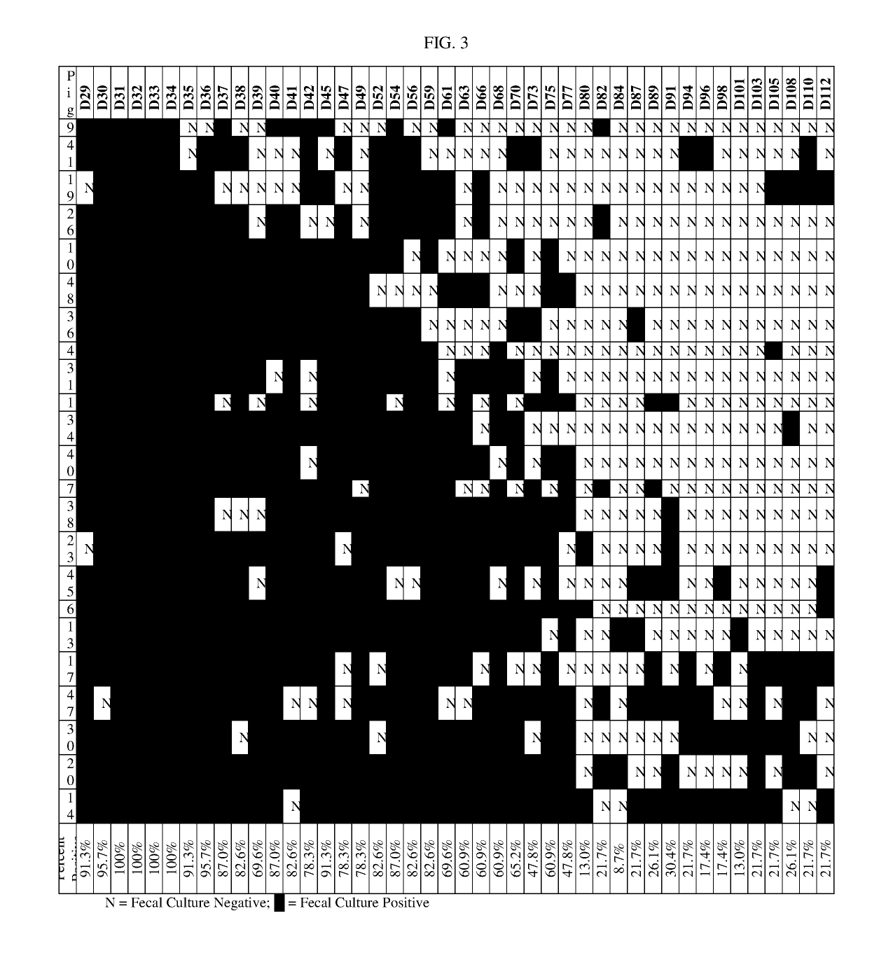

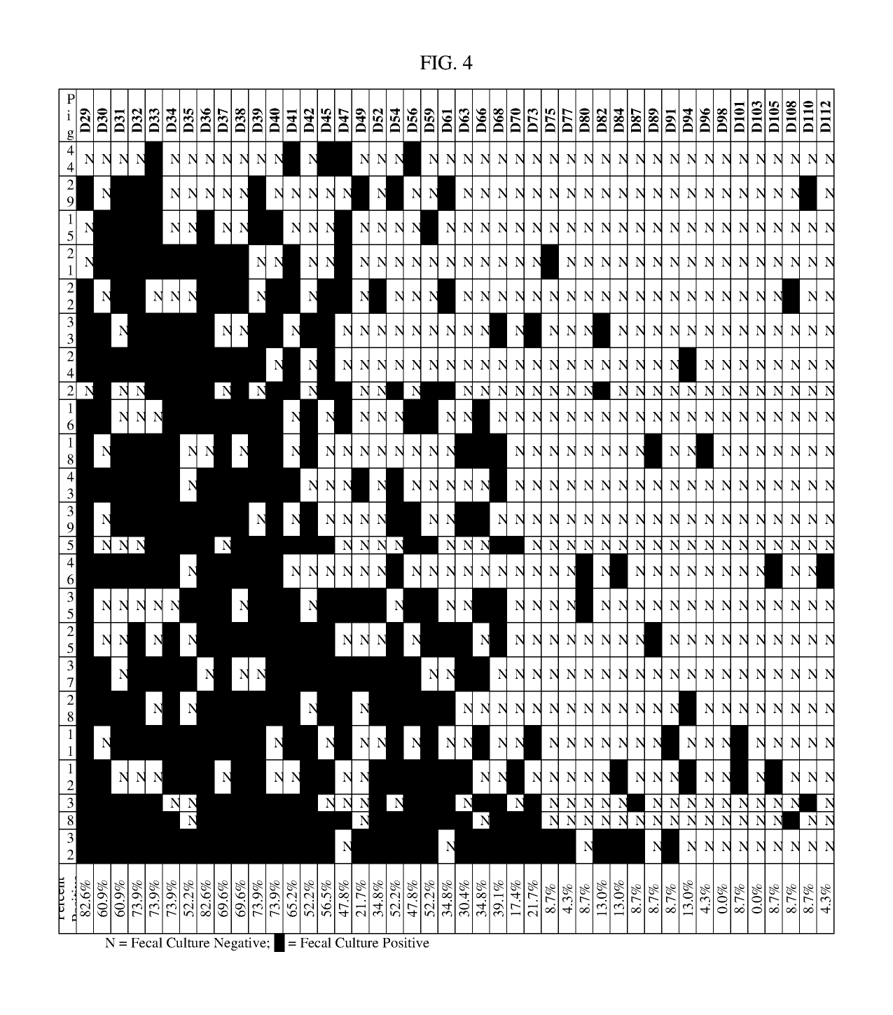

A pig was considered positive for shedding if group B Salmonella was cultured from a fecal sample post-challenge. Samples were collected from each pig daily for 14 days and approximately every other day for 70 days. The percent of pigs positive by treatment is shown over days post-challenge in FIG. 2. Individual pig data is shown in FIG. 3 for control pigs and FIG. 4 for vaccinated pigs.

Every pig in the T01 (control) group was fecal culture positive on D31, D32, D33 and D34. The percentage of pigs shedding each sampling day remained >69.6% until D61 (33 days post-challenge) and then was between 60.9% and 47.8% until D77 (49 days post-challenge). From D80 through D112, at least three pigs (13%) were positive every collection, with the exception of D84 when two pigs (8.7%) were positive.

While every pig in the T02 (vaccinated) group did have at least one positive culture during the post-challenge phase, the highest percentage of pigs shedding on any single day was 82.6% (on D29 and D36). Otherwise, the percentage of pigs shedding on any single day was between 52.2% and 73.9% from D30 to D45 (2 to 17 days post-challenge). After D45, the percentage of and number of T02 pigs decreased so that three or fewer pigs were positive from D75 to D112 (0% positive on D98 and D103).

While every pig in the T02 (vaccinated) group did have at least one positive culture during the post-challenge phase, the highest percentage of pigs shedding on any single day was 82.6% (on D29 and D36). Otherwise, the percentage of pigs shedding on any single day was between 52.2% and 73.9% from D30 to D45 (2 to 17 days post-challenge). After D45, the percentage of and number of T02 pigs decreased so that three or fewer pigs were positive from D75 to D112 (0% positive on D98 and D103).

For the 23 T01 (control) pigs that survived through the challenge phase (44 sampling days), the mean positive days was 26.22 days, with a minimum of 14 samples and a maximum of 39 samples (Table 3). For the 23 T02 (vaccinated) pigs that where challenged (44 sampling days), the mean number of positive days was 15.87 samples, with a minimum of 5 samples and a maximum of 31 samples. The MF for the number of positive results was 0.788 with a lower 95% confidence limit of 0.515.

TABLE 13
Number of Positive Samples by Treatment Group

| | | | | | | | Lower and |
|---|---|---|---|---|---|---|---|
| | | | | | | Mitigated | Upper |
| Group | N | Mean | Median | Min. | Max. | Fraction | 95CL |
| T01 | 23 | 26.22 | 26 | 14 | 39 | | |
| (Control) | | | | | | | |
| T02 | 23 | 15.87 | 15 | 5 | 31 | 0.788 | 0.515, 1.000 |
| (Vaccine) | | | | | | | |

N = number;
Min. = Minimum;
Max. = Maximum;
95CL = 95% Confidence Limit

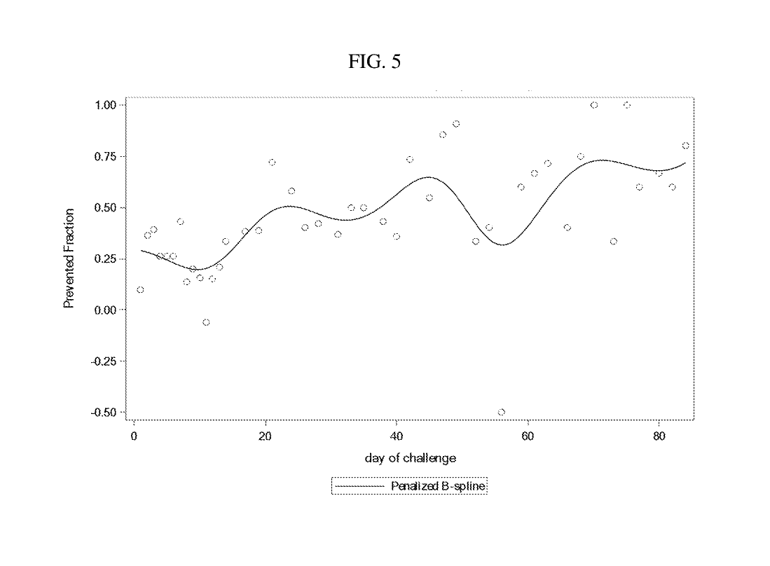

When considering the vaccine effect on a sampling basis, the prevented fraction (proportion affected in T01 minus proportion affected in T02)/(proportion affected in T01) can be utilized. The prevented fraction for each day was positive for 42 of the 44 collection days post-challenge (FIG. 5). Both days when the PF was negative were due to a difference of one pig: D39 was 16 T01 pigs versus 17 T02 pigs and D83 was two T01 pigs versus three T02 pigs.

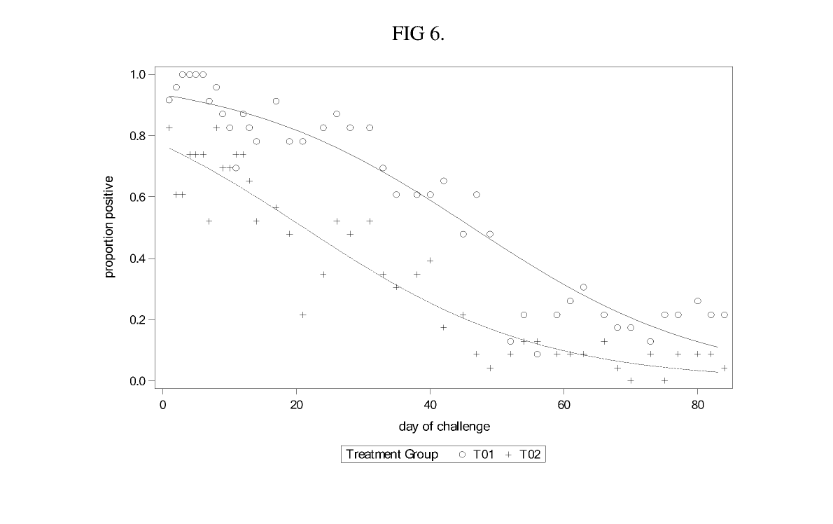

The probably of shedding was analyzed using the generalized linear mixed model (logistic model). The midpoint estimate for shedding in T01 (control) pigs was 46.31 days and for T02 (vaccinated) pigs as 21.09 days. The estimated treatment effect was 25.22 days, which was highly significant at P<0.0001. The logistical modelling for each treatment group is diagrammed in FIG. 6.

The first positive sample for all pigs was D29 or D30 with the exception of one T02 pig (#44) which was D33. The minimum duration was 48.0 days for T01 (control) pigs and 25.5 days for T02 (vaccinated) pigs (Table 14). Both groups had at least one pig with positive fecal samples on the final day of sampling (five pigs in T01 and 1 pig in T02). The MF was 0.288 with a lower confidence limit of −0.194.

TABLE 14
NUMBER OF POSITIE SAMPLES BY TREATMENT GROUP

| | | | | | | | Lower and |
|---|---|---|---|---|---|---|---|
| | | | | | | Mitigated | Upper |
| Group | N | Mean | Median | Min. | Max. | Fraction | 95CL |
| T01 | 23 | 69.74 | 74.0 | 48.0 | 84.0 | | |
| (Control) | | | | | | | |
| T02 | 23 | 59.17 | 62.0 | 25.5 | 84.0 | 0.288 | −0.194, |
| (Vaccine) | | | | | | | 0.683 |

N = number;
Min. = Minimum;
Max. = Maximum;
95CL = 95% Confidence Limit

The average body weights and ADWG for the vaccination and challenge phases are shown in Table 15. No differences in body weights or ADG were seen.

TABLE 15
LEAST SQUARE MEANS FOR BODY WEIGHTS BY
TREATMENT AND DAY WITH AVERAGE DAILY
WEIGHT GAIN (ADWB) FOR CHALLENGE PHASE

| | | | | | | | Challenge |
|---|---|---|---|---|---|---|---|
| | | | | | | | Phase |
| | | D-1 | | D27 | | D112 | ADWG |
| Group | n | (kg) | n | (kg) | n | (kg) | (kg/day) |
| Control | 24 | 5.66¹ | 24 | 21.65¹ | 23 | 96.88¹ | 0.885¹ |
| Vaccine | 24 | 5.58¹ | 23 | 21.05¹ | 23 | 96.60¹ | 0.889¹ |

¹P within column >0.05.

### DISCUSSION AND CONCLUSIONS

This vaccination-challenge study was considered valid as the T01 pigs remained healthy and fecal culture negative for vaccine strains prior to challenge. All pigs shed group B Salmonella in feces after challenge with 1.33×108 CFU/2-mL dose. Enteric pathogens have sporadic fecal shedding patterns as was observed in this study. While expectations are not that vaccination will eliminate shedding, the T02 (vaccine) group had a significant impact on Salmonella fecal shedding after challenge.

The determination for duration presents the data as continuous shedding through the challenge phase, which was not representative of the actual shedding pattern. Due to the sporadic nature of shedding later in the challenge phase, duration does not appear to be a relevant endpoint for measuring the vaccine's effect on the pattern of shedding after challenge. However, logistic model analysis quantifies the vaccines effect based on a statistically significant (P<0.0001) reduction of 25.22 days is estimated to achieve a time point where 50% of the animals are no longer shedding. Berends, B R et al 1996. “Identification and quantification of risk factors in animal management and transport regarding Salmonella spp. in pigs.” International Journal of Food Microbiology. Vol. 30, pp 37-53 and Kranker, S et al. 2003. “Longitudinal study of Salmonella enterica serotype Typhimurium infection in three Danish farrow-to-finish swine herds.” Journal of Clinical Microbiology. Vol. 41, No. 6, pp 2282-2288, both references hereby incorporated by reference. Both describe natural infection (shedding) in 80-100% of pigs the first weeks after arrival with considerable variation in duration between and within cohorts. The ability of the vaccine to accelerate reduction by nearly a month provides strong scientific application for producers to reliably and consistently reduce the duration of shedding in the field.

When considering the number of positive samples, the vaccine provided an important reduction in shedding when compared to the control group. The T02 (vaccine) group mean was 15.87 samples compared to the T01 (control) group mean of 26.22 samples for a MF of 0.788 (lower and upper 95% confidence limits of 0.5152 and 1.000). This reduction is further illustrated by the prevented fraction calculations, since the estimated prevented fraction was favorable for 42 of the 44 collection days post-challenge. Lo Fo Wong, D M A, et al. 2002. “Epidemiology and control measures for Salmonella in pigs and pork.” Livestock Production Science. Vol. 76, pp 215-222, hereby incorporated by reference, state that efforts to control Salmonella infection in pigs should include minimizing or preventing exposure to Salmonella, and this study demonstrates a statistical, clinically-relevant reduction in the number of days fecal samples were positive for Salmonella.

In summary, this study demonstrates a vaccine effect resulting in a consistently lower proportion of vaccinated pigs shedding throughout the challenge phase and a significant reduction in time for vaccinated pigs to achieve mid-point shedding. This supports a claim that the Salmonella Choleraesuis-Typhimurium Vaccine, Avirulent Live Culture of the present invention reduces Salmonella shedding when administered in drinking water at two weeks of age.

What is claimed is:

1. A method of reducing fecal shedding of Salmonella Typhimurium comprising:
administering an effective amount of a Salmonella Choleraesuis-Typhimurium vaccine or immunogenic composition to an animal in need thereof.

2. The method according to claim 1, wherein the animal is swine.

3. The method according to claim 1, wherein said immunogenic composition is administered orally.

4. The method according to claim 1, wherein said immunogenic composition is administered via drinking water.

5. The method according to claim 2, wherein said swine is at least 14 days of age.

6. The method according to claim 1, wherein said immunogenic composition is administered in 1 or 2 doses.

7. The method according to claim 6, wherein said immunogenic composition comprises one or more pharmaceutically acceptable carriers or excipients.

8. The method according to claim 7, wherein such one or more pharmaceutically acceptable carriers and/or excipients is one or more adjuvants.

9. A method for protecting a swine against diseases associated with Salmonella, comprising administering to swine, the immunogenic composition according to claim 1.

10. The immunogenic composition according to claim 1, wherein said immunogenic composition is an avirulent live culture.

11. A method according to claim 1, wherein a pig has reduced fecal shedding as compared to an unvaccinated pig.
